(19) United States
(12) Patent Application Publication
(10) Pub. No.: US 2025/0049956 A1
(43) Pub. Date: Feb. 13, 2025

(54) IN VIVO REPROGRAMMING OF PHOTORECEPTOR CELLS

(71) Applicant: CENTRE FOR EYE RESEARCH AUSTRALIA LTD, Victoria (AU)

(72) Inventor: Ching Bong WONG, East Melbourne (AU)

(21) Appl. No.: 18/721,144

(22) PCT Filed: Dec. 22, 2022
(86) PCT No.: PCT/AU2022/051573

§ 371 Date: Jun. 17, 2024

(30) Foreign Application Priority Data
Dec. 22, 2021 (AU) 2021904201

(51) Int. Cl. A61K 48/00; A61P 27/02; C12N 15/86

(52) U.S. Cl. CPC A61K 48/0058; A61P 27/02; C12N 15/86; C12N 2750/14143; C12N 2750/14145; C12N 2750/14152; C12N 2750/14171

(57) ABSTRACT

The invention relates to in vivo methods and compositions for converting one cell type to another cell type. In particular, the invention relates to transdifferentiation of a cell to a photoreceptor cell, preferably a rod photoreceptor cell. In one aspect, the invention provides an in vivo method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein the source cell is a glial cell, the target cell is a photoreceptor cell; and the transcription factors are one or more of those selected from ASCLI, NEURODI, NRL, NR2E3, RAX, RORB, OTX2, CRX and PAX6.

### FIELD OF THE INVENTION

The invention relates to in vivo methods and compositions for converting one cell type to another cell type. In particular, the invention relates to transdifferentiation of a cell to a photoreceptor cell, preferably a rod photoreceptor cell.

### RELATED APPLICATION

This application claims priority from Australian provisional application 2021904201, the entire contents of which are hereby incorporated by reference in their entirety.

### BACKGROUND OF THE INVENTION

Cell-based regenerative therapy requires the generation of specific cell types for replacing tissues damaged by injury, disease or age. Embryonic stem cells (ESC) have the potential to differentiate in every cell type from the (human) body and have therefore been extensively studied as a source for replacement therapy. However, ESC cannot be derived in a patient-specific fashion since they are established from cultured blastocysts. Therefore, immune rejection and ethical concerns are the main barriers that prevent the transfer of the ESC technology, and in particular of human ESC technology, to clinical applications.

Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated.

Trans-differentiation, the process of converting from one cell type to another without going through a pluripotent state, may have great promise for regenerative medicine but has yet to be reliably applied. Although it may be possible to switch the phenotype of one somatic cell type to another, the elements required for conversion are difficult to identify and in most instances unknown. The identification of factors to directly reprogram the identity of cell types is currently limited by, amongst other things, the cost of exhaustive experimental testing of plausible sets of factors, an approach that is inefficient and unscaleable.

Photoreceptor cells, also known simply as photoreceptors, are light-sensing cells within the retina that form the basis of human vision. The degeneration of photoreceptors is a central hallmark of many blinding diseases, including retinitis pigmentosa, age-related macular degeneration, choroideremia and diabetic retinopathy. These diseases affect millions worldwide and results in a significant socio-economic burden on our healthcare system. Critically, there is no cure to blindness once the photoreceptors in the eye are lost. Also, at the late stages of these retinal degenerative diseases, there are often insufficient remaining photoreceptors that can be targeted for pharmacological treatment. In this regard, regenerative medicine represents a highly attractive approach to address this issue.

There is a need for a new and/or improved method for generating cells and cell populations, particularly photoreceptor cells, for use in research and therapeutic applications.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to in vivo methods and compositions for direct reprogramming (i.e. transdifferentiation or cellular reprogramming) of a source cell to a cell having characteristics of a photoreceptor cell, particularly a rod photoreceptor cell.

In one aspect, the present invention provides an in vivo method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein:
the source cell is a glial cell,
the target cell is a photoreceptor cell or photoreceptor-like cell; and
the transcription factors are one or more of those selected from ASCL1, NEUROD1, NRL, NR2E3, RAX, RORB, OTX2, CRX and PAX6.

Preferably, the glial cell is selected from the group consisting of a Müller glial (MG) cell, an astrocyte and a microglia. The glial cell may be a retinal glial cell.

In any aspect or embodiment, the photoreceptor cells are rod photoreceptor cells.

In one aspect, the present invention provides a nucleic acid or vector comprising an expression construct encoding one or more transcription factors selected from ASCL1, NEUROD1, NRL, NR2E3, RAX, RORB, OTX2, CRX and PAX6 or biologically active fragments or variants thereof.

In any aspect of a method, use or nucleic acid of the invention described herein, the transcription factors, or biologically active fragments or variants thereof, are least two of: ASCL1, NEUROD1, NRL, NR2E3, RAX, RORB, OTX2, CRX and PAX6; at least three of ASCL1, NEUROD1, NRL, NR2E3, RAX, RORB, OTX2, CRX and PAX6; at least four of ASCL1, NEUROD1, NRL, NR2E3, RAX, RORB, OTX2, CRX and PAX6; at least five of ASCL1, NEUROD1, NRL, NR2E3, RAX, RORB, OTX2, CRX and PAX6; at least six of ASCL1, NEUROD1, NRL, NR2E3, RAX, RORB, OTX2, CRX and PAX6; at least 7 of ASCL1, NEUROD1, NRL, NR2E3, RAX, RORB, OTX2, CRX and PAX6; at least 8 of ASCL1, NEUROD1, NRL, NR2E3, RAX, RORB, OTX2, CRX and PAX6; or all 9 of ASCL1, NEUROD1, NRL, NR2E3, RAX, RORB, OTX2, CRX and PAX6, or biologically active fragments or variants thereof.

In any aspect of a method, use or nucleic acid of the invention described herein, the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3;
(n) NR2E3, OTX2, RAX and NEUROD1;
(o) OTX2;
(p) ASCL1, NRL, NR2E3 and CRX;
(q) ASCL1, NEUROD1, NRL and RORB;
(r) ASCL1, NEUROD1, NRL and OTX2;
(s) RAX;
(t) OTX2, RAX and NEUROD1;
(u) ASCL1, NRL, NR2E3 and RAX;
(v) RORB;
(w) NR2E3, OTX2, CRX, RAX and NEUROD1;
(x) ASCL1;
(y) NEUROD1;
(z) PAX6;
(aa) NEUROD1, CRX, NR2E3 and RAX;
(bb) CRX;
(cc) ASCL1, NRL, RORB and OTX2;
(dd) NRL;
(ee) ASCL1, NEUROD1, NRL, RAX and NR2E3;
(ff) ASCL1, NEUROD1, NRL, NR2E3 and CRX;
(gg) ASCL1, NRL, RORB and CRX;
(hh) NR2E3;
(ii) CRX, RAX and NEUROD1;
(jj) CRX, OTX2 and NRL; or
(kk) NR2E3 and PAX6.

In any aspect of a method, use or nucleic acid of the invention described herein, the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3;
(n) NR2E3, OTX2, RAX and NEUROD1;
(0) OTX2;
(p) ASCL1, NRL, NR2E3 and CRX;
(q) ASCL1, NEUROD1, NRL and RORB;
(r) ASCL1, NEUROD1, NRL and OTX2;
(s) RAX;
(t) OTX2, RAX and NEUROD1;
(u) ASCL1, NRL, NR2E3 and RAX;
(v) RORB;
(w) NR2E3, OTX2, CRX, RAX and NEUROD1; or
(x) NR2E3 and PAX6.

In any aspect of a method, use or nucleic acid of the invention described herein, the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3; or
(n) NR2E3 and PAX6.

In any aspect of a method, use or nucleic acid of the invention described herein, the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX; or
(d) NR2E3 and PAX6.

In any aspect of a method, use or nucleic acid of the invention described herein, the transcription factors, or biologically active fragments or variants thereof, are:
(a) ASCL1, NEUROD1 and NRL; or
(b) NR2E3 and PAX6.

In any embodiment, the nucleic acid or vector comprises or consists of an expression construct. Preferably, the expression construct comprises one or more features of an AAV, or rAAV, vector of the invention as described herein.

In another aspect, the present invention also provides a recombinant vector comprising an expression construct as described herein. The recombinant vector may be a recombinant AAV (rAAV) vector.

The promoter in the expression construct, or any other aspect of the invention described herein, may be any nucleotide sequence that is capable of inducing RNA polymerase to bind to and transcribe the coding sequence. The promoter may be a ubiquitous promoter or a glial cell-specific promoter

In one preferred embodiment, the promoter is the CAG promoter. The CAG promoter preferably comprises the cytomegalovirus (CMV) early enhancer element, the promoter, the first exon and the first intron of chicken beta-actin (CBA) gene and the splice acceptor of the rabbit beta-globin gene.

In one embodiment the nucleotide sequence encoding the CMV early enhancer element is 245 bp long, and is referred to herein as SEQ ID NO: 1. Preferably, the CMV early enhancer element comprises a nucleotide sequence substantially as set out in SEQ ID NO: 1, or a fragment or variant thereof.

In one embodiment the nucleotide sequence encoding the GFAP promoter is 681 bp long, and is referred to herein as SEQ ID NO: 2. Preferably, the promoter comprises a nucleotide sequence substantially as set out in SEQ ID NO: 2, or a fragment or variant thereof.

In one embodiment the nucleotide sequence encoding the first intron of chicken-beta actin gene (CBA) is 408 bp long, and is referred to herein as SEQ ID NO: 3. Preferably, the first intron of CBA comprises a nucleotide sequence substantially as set out in SEQ ID NO: 3, or a fragment or variant thereof.

Preferably, the expression construct, or any other aspect of the invention described herein, comprises a nucleotide sequence encoding a Kozak sequence, which enhances transcription factor expression or a biologically active fragment or variant thereof. Preferably, the Kozak coding sequence is disposed 5′ of the nucleotide sequence encoding the one or more sets of transcription factors as described herein, including in (a) through to (jj) above, and Table 3 below, or biologically active fragments or variants thereof.

In one embodiment the nucleotide sequence encoding the Kozak sequence is 10 bp long, and is referred to herein as SEQ ID NO: 4. Preferably, the Kozak sequence comprises a nucleotide sequence substantially as set out in SEQ ID NO: 4, or a fragment or variant thereof.

Preferably, the expression construct, or any other aspect of the invention described herein, comprises a nucleotide sequence encoding a Woodchuck Hepatitis Virus Post-transcriptional Regulatory Element (WPRE), which enhances expression of one or more sets of transcription factors as described herein, including in (a) through to (jj) above, and Table 3 below or biologically active fragments or variants thereof. Preferably, the WPRE coding sequence is disposed 3′ of the nucleotide sequence encoding the one or more sets of transcription factors as described herein, including in (a) through to (jj) above, and Table 3 below or biologically active fragments or variants thereof.

In one embodiment the nucleotide sequence encoding WPRE is 593 bp long, and is referred to herein as SEQ ID NO: 5. Preferably, the WPRE comprises a nucleotide sequence substantially as set out in SEQ ID NO: 5, or a fragment or variant thereof.

Preferably, the expression construct, or any other aspect of the invention described herein, comprises a nucleotide sequence encoding a bovine growth hormone (bGH) polyA tail. Preferably, the bGH polyA tail coding sequence is disposed 3′ of the nucleotide sequence encoding the one or more sets of transcription factors as described herein, including in (a) through to (jj) above, and Table 3 below or biologically active fragments or variants thereof, and preferably 3′ of the WPRE coding sequence.

In one embodiment, the nucleotide sequence encoding the bovine growth hormone (bGH) polyA tail is 269 bp long, and is referred to herein as SEQ ID NO: 6. Preferably, the bovine growth hormone polyA tail comprises a nucleotide sequence substantially as set out in SEQ ID NO: 6, or a fragment or variant thereof.

In any embodiment, the expression construct comprises AAV Inverted Terminal Repeats (ITRs), for example AAV ITRs flanking the nucleotide sequence encoding the one or more sets of transcription factors as described herein, including in (a) through to (ee) above, and Table 3 below or biologically active fragments or variants thereof.

Preferably, the expression construct, or any other aspect of the invention described herein, comprises left and/or right ITRs. Preferably, each ITR is disposed at the 5′ and/or 3′ end of the construct.

In one embodiment, the nucleotide sequence of the left ITR is represented herein as SEQ ID NO: 7.

In one embodiment, the nucleotide sequence of the right ITR is represented herein as SEQ ID NO: 8.

In another aspect, the present invention also provides an adeno-associated viral (AAV) vector, lentiviral vector, baculoviral vector or mRNA (e.g. synthetic mRNA) comprising a nucleotide sequence encoding the one or more sets of transcription factors as described herein, including in (a) through to (jj) above, and Table 3 below or biologically active fragments or variants thereof. Typically, the nucleotide sequence encoding the one or more sets of transcription factors as described herein, including in (a) through to (jj) above, and Table 3 below or biologically active fragments or variants thereof is flanked by two AAV Inverted Terminal Repeats (ITRs).

In any embodiment, the AAV vector, lentiviral vector or baculoviral vector is recombinant, synthetic, purified, or substantially purified.

In some embodiments, the AAV vector is a recombinant (rAAV) vector. The rAAV may be a naturally occurring vector or a vector with a hybrid AAV serotype. The rAAV may be AAV-1, AAV-2, AAV-3, AAV-4, AAV-5, AAV-6, AAV-7, AAV-8, AAV-9, AAV-10, AAV-11, AAV-12, AAV-13, ShH10 and ShH10Y.

The recombinant AAV vector may be a bioengineered vector. The rAAV may be Anc80, DJ, DJ/8, KP1, KP2, KP3, LK01, LK02, LK03, LK19, NP6, NP22, NP40, NP59, NP66, NP84, NP94, rh10, 2i8, 7m8, PHP.eB and AAV2 Retro.

The recombinant vector may be SYD01, SYD03, SYD09, HRS1, HRS19, HRS5, CD15, T33, CMRI-01, CMRI-02, CMRI-03, CMRI-04, CMRI-05, CMRI-06, CMRI-07 and CMRI-08.

Preferably, however, the rAAV ShH10 or ShH10Y.

Advantageously, ShH10 and ShH10Y, derived from an AAV6 parent serotype, is capable of efficient, selective Müller cell infection through intravitreal injection. ShH10 and ShH10Y also shows significantly improved transduction relative to AAV2 (>60%) and AAV6.

The term “recombinant (rAAV) vector” as used herein means a recombinant AAV-derived nucleic acid containing at least one terminal repeat sequence.

Preferably, the expression construct, recombinant plasmid vector or any other aspect of the invention described herein comprises at least one stuffer sequence, preferably one or more than one of the following stuffer sequences described below. Preferably, the recombinant plasmid vector comprises a first, second, third, fourth, fifth, sixth and/or seventh stuffer sequence, preferably wherein the first, second, third, fourth, fifth, sixth and seventh stuffer sequences are as described herein (e.g. SEQ ID NO: 9-15.

In one embodiment, the first stuffer sequence is represented herein as SEQ ID No: 9.

In one embodiment, the second stuffer sequence is represented herein as SEQ ID No: 10.

In one embodiment, the third stuffer sequence is represented herein as SEQ ID No: 11.

In one embodiment, the fourth stuffer sequence is represented herein as SEQ ID No: 12.

In one embodiment, the fifth stuffer sequence is represented herein as SEQ ID No: 13.

In one embodiment, the sixth stuffer sequence is represented herein as SEQ ID No: 14.

In one embodiment, the sixth stuffer sequence is represented herein as SEQ ID No: 15.

Preferably, the expression construct, recombinant plasmid vector or any other aspect of the invention described herein comprises an antibiotic resistance gene. Typically, the antibiotic resistance gene is a nucleotide sequence encoding a kanamycin resistance gene.

In one embodiment, the nucleotide sequence encoding the kanamycin resistance gene is 816 bp long, and is referred to herein as SEQ ID NO: 16.

Preferably, the expression construct, recombinant plasmid vector or any other aspect of the invention described herein comprises a pUC origin. Typically, nucleotide sequence encoding the pUC origin is 668 bp long, and is referred to herein as SEQ ID No: 17.

In any embodiment, the AAV vector, lentiviral vector, baculoviral vector or synthetic mRNA further comprises one or more regulatory sequences (e.g. promoter) that allows, or causes, expression of the one or more sets of transcription factors as described herein, including in (a) through to (jj) above, and Table 3 below or biologically active fragments or variants thereof in glial cells, preferably retinal glial cells.

Preferably, the promoter is a ubiquitous promoter or an glial cell-specific promoter. In any embodiment, the nucleotide sequence encoding the one or more sets of transcription factors as described herein, including in (a) through to (jj) above, and Table 3 below or biologically active fragments or variants thereof is operably linked to the promoter.

Examples of ubiquitous promoters include a CAG promoter. The CAG promoter preferably comprises the cytomegalovirus (CMV) early enhancer element, the promoter, the first exon and the first intron of chicken beta-actin (CBA) gene and the splice acceptor of the rabbit beta-globin gene.

Examples of glial cell-specific promoters include the promoters for GFAP, GLAST and RLBP1 genes and/or combinations of glial cell-specific transcription factor regulatory elements.

In some embodiments, the AAV vector comprises a CMV promoter, for example as described herein. In some embodiments, the AAV vector comprises a Kozak sequence, for example as described herein. In some embodiments, the vector comprises one or more ITR sequence flanking the vector portion encoding the one or more sets of transcription factors as described herein, including in (a) through to (ee) above, and Table 3 below or biologically active fragments or variants thereof, for example as described herein. In some embodiments, the vector comprises a polyadenylation sequence. In some embodiments, the vector comprises a selective marker. Preferably, the selective marker is an antibiotic-resistance gene, such as an ampicillin-resistance gene or a kanamycin-resistance gene.

In any embodiment, the ITR, or each ITR if two or more, is a wildtype AAV2 ITR sequence, or ITR as described herein.

In one embodiment, the present invention provides a recombinant adeno-associated virus (AAV) vector comprising a nucleic acid comprising, in 5′ to 3′ order:
(a) a 5′ AAV ITR, for example SEQ ID NO: 7 or 8;
(b) a CMV enhancer, for example SEQ ID NO: 1;
(c) an glial cell-specific promoter;
(d) a transgene encoding the one or more sets of transcription factors as described herein, including in (a) through to (ee) above, and Table 3 below or biologically active fragments or variants thereof;
(e) a WPRE, for example SEQ ID NO: 5;
(f) a Bovine Growth Hormone polyA signal tail, for example SEQ ID NO: 6; and
(g) a 3 ′AAV ITR, for example SEQ ID NO: 7 or 8.

In another aspect, the present invention provides a recombinant adeno-associated virus (rAAV) comprising:
(i) an AAV capsid protein; and
(ii) an AAV vector of the invention as described herein.

In one embodiment, the AAV capsid protein is a ShH10 or ShH10Y capsid protein.

In any embodiment, the rAAV may be a AAV variant or mutant as described herein.

In another aspect, the present invention provides a pharmaceutical composition comprising an nucleic acid of the invention as described herein, a vector, preferably an AAV vector of the invention as described herein, or a recombinant AAV of the invention as described herein, and a pharmaceutically acceptable carrier, diluent or excipient.

In another aspect, the present invention provides a plasmid comprising nucleic acid comprising an expression construct of the invention as described herein, or a vector, preferably an AAV vector of the invention as described herein.

In another aspect, the present invention provides a Baculovirus vector comprising a nucleic acid of the invention as described herein.

In another aspect, the present invention provides a cell comprising:
(i) a first vector encoding one of more adeno-associated virus rep protein and/or one or more adeno-associated virus cap protein; and
(ii) a second vector comprising a nucleotide sequence encoding the one or more sets of transcription factors as described herein, including in (a) through to (ee) above, and Table 3 below or biologically active fragments or variants thereof.

In one embodiment, the first vector is a plasmid and the second vector is a plasmid. In another embodiment, the first vector is a Baculovirus vector and the second vector is a Baculovirus vector.

Typically, the cell is a mammalian cell, preferably the mammalian cell is a HEK293 cell. Alternatively, the cell is an insect cell, preferably the insect cell is a SF9 cell.

In another aspect, the present invention provides a method of producing an AAV of the invention as described herein, the method comprising:
(i) delivering to a cell a first vector encoding one or more adeno-associated virus rep protein and/or one or more adeno-associated cap protein, and a recombinant AAV vector comprising a expression cassette comprising a nucleotide sequence that encodes the one or more sets of transcription factors as described herein, including in (a) through to (ee) above, and Table 3 below or biologically active fragments or variants thereof;
(ii) culturing the cells under conditions allowing for packaging the AAV; and
(iii) harvesting the cultured host cell or culture medium for collection of the AAV.

In another aspect, the present invention provides a method of decreasing progression of or ameliorating vision loss associated with or cause by degeneration, or loss, of rod photoreceptor cells in a subject, the method comprising administering to the subject an nucleic acid of the invention as described herein, a vector, preferably an AAV vector, of the invention as described herein, a recombinant AAV of the invention as described herein, or a pharmaceutical composition of the invention as described herein, thereby of decreasing progression of or ameliorating vision loss associated with or cause by degeneration, or loss, of rod photoreceptor cells.

In another aspect, the present invention provides use of an nucleic acid of the invention as described herein, a vector, preferably an AAV vector, of the invention as described herein, a recombinant AAV of the invention as described herein, or a pharmaceutical composition of the invention as described herein, in the manufacture of a medicament for decreasing progression of or ameliorating vision loss associated with or cause by degeneration, or loss, of rod photoreceptor cells in a subject.

In another aspect, the present invention provides a nucleic acid of the invention as described herein, a vector, preferably, an AAV vector of the invention as described herein, a recombinant AAV of the invention as described herein, or a pharmaceutical composition of the invention as described herein, for use in decreasing progression of or ameliorating vision associated with or cause by degeneration, or loss, of rod photoreceptor cells in a subject.

In any aspect, preferably the subject is a human.

In any aspect or embodiment, the condition associated with or cause by degeneration, or loss, of rod photoreceptor cells may also be referred to as a rod cell disorder. The degeneration, or loss, of rod photoreceptor cells is associated with or causes changes in vision, typically a reduction in vision.

In some embodiments, the rod cell disorder is a retinal degenerative disorder. In some embodiments, the rod cell disorder is a macular dystrophy or retinal dystrophy. The macular dystrophy may be selected from the group consisting of Stargardt's macular dystrophy, rod dystrophy (including rod-cone dystrophy and cone-rod dystrophy), Spinocerebellar ataxia type 7, and Bardet-Biedl syndrome-1. Preferably, the macular dystrophy is Stargardt's macular dystrophy or rod-cone dystrophy. In some embodiments, the rod cell disorder is a vision disorder of the central macula or a retinal dystrophy. In certain embodiments, vision disorder of the central macula or retinal dystrophy is selected from the group consisting of age-related macular degeneration, macular telangiectasia, retinitis pigmentosa, diabetic retinopathy, retinal vein occlusions, glaucoma, choroideremia, Sorsby's fundus dystrophy, adult vitelliform macular dystrophy, Best's disease, Leber's congenital amaurosis, and X-linked retinoschisis. Preferably, the vision disorder is retinitis pigmentosa, age-related macular degeneration or diabetic retinopathy.

In any embodiment, the subject has been diagnosed with a condition associated with or cause by degeneration, or loss, of rod photoreceptor cells as described herein. Preferably, the individual has been diagnosed with a rod dystrophy. The individual may have been diagnosed with progressive rod dystrophy or stationary rod dystrophy. The rod dystrophy may be a rod-cone dystrophy or a cone-rod dystrophy.

In some such embodiments, the method further comprises detecting a change in the condition or disorder symptoms. Including any symptom described herein. In some such embodiments, the change comprises a stabilization in the health of the existing or reprogrammed rod cells and/or a reduction in the rate of visual acuity loss of the subject. In certain such embodiments, the change comprises an improvement in in the visual acuity of the subject.

In some such embodiments, the method further comprises detecting a change in the condition or disorder symptoms, wherein the change comprises an increase in the vision of a subject.

In any aspect of the present invention, the nucleic acid of the invention as described herein, a vector, preferably an AAV vector, of the invention as described herein, a recombinant AAV of the invention as described herein, or a pharmaceutical composition of the invention as described herein, is administered to the subject by retinal administration. Exemplary retinal administration is retinal injection (e.g. subretinal or intravitreal injection) into an affected eye of said subject.

In another aspect, the present invention provides for a composition comprising any of the AAV vectors or rAAV of the invention as disclosed herein and a pharmaceutically acceptable carrier, excipient or diluent.

In any aspect, increasing the amount of one or more transcription factors, or biologically active fragments or variants thereof, in a source cell includes transfecting said source cell with one or more nucleic acids for increasing the expression of one or more genes encoding one or more transcription factors.

Preferably, the at least one characteristic of the photoreceptor cell is up-regulation of any one or more target cell markers and/or change in cell morphology. Relevant markers are described herein and known to those in the art. Exemplary markers for the photoreceptor cells include:
RHO;
MYO7A;
PDE6B;
CNGB1;
NR2E3;
ROM1;
MEF2C;
ELOVL4;
NRL;
GNAT1;
CNGA1;
SAG;
GNGT1;
an electrophysiological response in a scotopic condition, for example, as described in the Examples. Markers RHO, MYO7A, PDE6B, CNGB1, NR2E3, ROM1, MEF2C, and ELOVL4 are also markers of rod photoreceptor cells.

Additional examples of photoreceptor markers include the opsins that are light-detecting molecules. For example, rhodopsin (rod photoreceptor cells), red/green opsin (cone photoreceptor cells), blue opsin (cone photoreceptor cells), and recoverins (rod photoreceptor cells, cone photoreceptor cells).

In any aspect, the combination of transcription factors selected from ASCL1, NEUROD1, NRL, NR2E3, RAX, RORB, OTX2, CRX and PAX6 results in a photoreceptor, or photoreceptor-like, cells with a fold increase in RHO mRNA expression of equal to, or greater than, 2, 3, 4, 5, 6, 7, 8, 9, 10, 15, 20, 25, 30, 35, 40, 45, 50, 55, 60, 65, 70 or 75 fold compared to the RHO expression in the source cell type. Preferably, the fold increase is equal to, or greater than, 4, 5, 6, 7, 8, 9, 10, 15, 20, 25, 30, 35, 40, 45, 50, 55, 60, 65, 70 or 75 fold compared to the RHO expression in the source cell type. Most preferably, the fold increase is equal to, or greater than, 10, 15, 20, 25, 30, 35, 40, 45, 50, 55, 60, 65, 70 or 75 fold compared to the RHO expression in the source cell type.

In any aspect of the present invention, the source cell is a human cell. Where the source cell is a Müller glial cell, it may be a human Müller glial cell.

The present invention also relates to kits for producing a cell exhibiting at least one characteristic of a photoreceptor cell as disclose herein. In some embodiments, a kit comprises one or more nucleic acids having one or more nucleic acid sequences encoding a transcription factor described herein or biologically active fragments or variants thereof, including the specific combinations referred to in (a) to (jj) herein. Preferably, the kit can be used with a source cell referred to herein. In some embodiments, the kit further comprises instructions for reprogramming a source cell to a cell exhibiting at least one characteristic of a photoreceptor cell according to the methods as disclosed herein. Preferably, the present invention provides a kit when used in a method of the invention described herein.

Typically, the gene expression, or amount, of a transcription factor as described herein is increased by introducing at least one nucleic acid comprising a nucleotide sequence encoding a transcription factor, or encoding a functional fragment thereof, in the cell. Preferably, the nucleotide sequence encoding a transcription factor is at least 70%, 80%, 85%, 90%, 91%, 92%, 93%, 94%, 95%, 96%, 97%, 98%, or 99% identical to a sequence with an accession number listed in Table 1.

In any aspect of the present invention, the method as described herein may have one or more, or all, steps performed in vivo.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. A) Fold change gene expression following CRISPR activation to induce expression of 9 candidate transcription factors in human MG cells (MIOMI) using single sgRNA, or multiplex expression of 9 sgRNA. n=3, error bars=SEM. B) RHO mRNA expression (fold change relative to mock) is shown for 125 reprogramming experiments to identify transcription factor cocktails that reprogram human MG cells into induced photoreceptor cells (iPH) (Arrows indicate: ANNr=ASCL1+NEUROD1+NRL; ANrR=ASCL1+NRL+RAX; 9 genes=ASCL1+NEUROD1+CRX+OTX2+NRL+RAX+RORB+NR2E3PAX6). Results represent mean of technical triplicates+SEM. C) RHO mRNA expression (fold change relative to mock) for selected reprogramming conditions as listed in Table 3. Nr2P was tested using a transgene overexpression system.

FIG. 2. A-B) Fluorescence microscopy images of derived iPH produced according to the invention. A) Staining with RHO and DAPI indicates that the iPH are positive for rod marker RHO. B) Staining with rod marker PDE6B also indicates that the derived iPH are positive for this marker. C) Violin plot of multielectrode array of mock control and iPH. Pre-illumination and post-illumination (left- and right-hand sides, respectively, for mock and iPH samples), demonstrate that the iPH display functional electrophysiological responses following light stimulation.

FIG. 3. A) Results of single cell RNAseq indicate the presence of an iPH subpopulation (arrow) in which all 9 rod markers are upregulated. C1-C8 represent clusters identified by single cell transcriptomics and represent subpopulations within the reprogrammed culture. B) Principal component analysis of transcriptomics is expected to induce a transcriptional shift from MG cells to rods. C) Using a single cell human retinal gene atlas as a benchmark, the pilot scRNAseq data captured various reprogramming stages of iPH from MG cells to rod photoreceptors specifically (marked line).

FIG. 4. A) Schematic of in vivo reprogramming testing in a rat retinitis pigmentosa (RP) model with photoreceptor degeneration (P23H3). Intravitreal injection of adeno-associated viruses (AAV) carrying iPH genes were performed in P23H3 rats, and visual response were analysed using electroretinogram (ERG) 4 weeks after treatment. B) Schematic of AAV vector used to deliver the iPH gene (Ascl, Neurod1 or Nrl) driven by the Muller glia (MG)-specific promoter GFAP, and generated using the MG-specific targeting AAV serotype ShH10Y.

FIG. 5. ERG analysis of P23H3 rats following injection of AAV carrying iPH genes: (A-B) Ascl1+Neurod1+Nrl (ANNr) or (C-D) Nr2e3+Pax6 (Nr2P), highlighting the functional improvement in visual response following AAV delivery of iPH genes. The a-wave depicting photoreceptor function (A,C) and the b-wave depicting bipolar function (B,D) were normalised before and after treatment for the individual eye. Naïve (untreated) controls and sham controls with PBS injection were used as negative controls. **: p<0.01, ***: p<0.001; ****: p<0.0001.

FIG. 6. Immunohistochemistry analysis showed localised increase in the thickness of the outer nuclear layer (ONL) in P23H3 rats (marked by white arrows) following treatment with AAV delivery of Ascl1+Neurod1+Nrl (ANNr) or Nr2e3+Pax6 (Nr2P), compared to sham control. DAPI was used as a nuclear counterstain together with various retinal markers: CRALBP (MG cells), CRX (photoreceptors), RHO (rods). Scale bar=50 um.

Sequence listing

| | SEQ | |
|---|---|---|
| Descrip- | ID | Nucleotide or amino |
| tion | NO | acid sequence |
| CMV early | 1 | CCATTGACGTCAATAATGACGTATGTTCCCATAGT |
| enhancer | | AACGCCAATAGGGACTTTCCATTGACGTCAATGGG |
| element | | TGGAGTATTTACGGTAAACTGCCCACTTGGCAGTA |
| | | CATCAAGTGTATCATATGCCAAGTACGCCCCCTAT |
| | | TGACGTCAATGACGGTAAATGGCCCGCCTGGCATT |
| | | ATGCCCAGTACATGACCTTATGGGACTTTCCTACT |
| | | TGGCAGTACATCTACGTATTAGTCATCGCTATTAC |
| Promoter | 2 | AACATATCCTGGTGTGGAGTAGGGGACGCTGCTCT |
| | | GACAGAGGCTCGGGGGCCTGAGCTGGCTCTGTGAG |
| | | CTGGGGAGGAGGCAGACAGCCAGGCCTTGTCTGCA |
| | | AGCAGACCTGGCAGCATTGGGCTGGCCGCCCCCCA |
| | | GGGCCTCCTCTTCATGCCCAGTGAATGACTCACCT |
| | | TGGCACAGACACAATGTTCGGGGTGGGCACAGTGC |
| | | CTGCTTCCCGCCGCACCCCAGCCCCCCTCAAATGC |
| | | CTTCCGAGAAGCCCATTGAGCAGGGGGCTTGCATT |
| | | GCACCCCAGCCTGACAGCCTGGCATCTTGGGATAA |
| | | AAGCAGCACAGCCCCCTAGGGGCTGCCCTTGCTGT |
| | | GTGGCGCCACCGGGGGTGGAGAACAAGGCTCTATT |
| | | CAGCCTGTGCCCAGGAAAGGGGATCAGGGGATGCC |
| | | CAGGCATGGACAGTGGGTGGCAGGGGGGGAGAGGA |
| | | GGGCTGTCTGCTTCCCAGAAGTCCAAGGACACAAA |
| | | TGGGTGAGGGGAGAGCTCTCCCCATAGCTGGGCTG |
| | | CGGCCCAACCCCACCCCCTCAGGCTATGCCAGGGG |
| | | GTGTTGCCAGGGGCACCCGGGCATCGCCAGTCTAG |
| | | CCCACTCCTTCATAAAGCCCTCGCATCCCAGGAGC |
| | | GAGCAGAGCCAGAGCAGGTTGGAGAGGAGACGCAT |
| | | CACCTCCGCTGCTCGC |
| First | 3 | GGAGTCGCTGCGCGCTGCCTTCGCCCCGTGCCCCG |
| intron | | CTCCGCCGCCGCCTCGCGCCGCCCGCCCCGGCTCT |
| of | | GACTGACCGCGTTACTCCCACAGGTGAGCGGGCGG |
| chicken- | | GACGGCCCTTCTCCTCCGGGCTGTAATTAGCGCTT |
| beta | | GGTTTAATGACGGCTTGTTTCTTTTCTGTGGCTGC |
| actin | | GTGAAAGCCTTGAGGGGCTCCGGGAGGGCCCTTTG |
| gene | | TGCGGGGGGAGCGGCTCGGGGCTGTCCGCGGGGGG |
| (CBA) | | ACGGCTGCCTTCGGGGGGGACGGGGCAGGGCGGGG |
| | | TTCGGCTTCTGGCGTGTGACCGGCGGCTCTAGAGC |
| | | CTCTGCTAACCATGTTCATGCCTTCTTCTTTTTCC |
| | | TACAGCTCCTGGGCAACGTGCTGGTTATTGTGCTG |
| | | TCTCATCATTTTGGCAAAGAATT |
| Kozak | 4 | GGCACCATGG |
| sequence | | |
| WPRE | 5 | ATCGATAATCAACCTCTGGATTACAAAATTTGTGA |
| sequence | | AAGATTGACTGGTATTCTTAACTATGTTGCTCCTT |
| | | TTACGCTATGTGGATACGCTGCTTTAATGCCTTTG |
| | | TATCATGCTATTGCTTCCCGTATGGCTTTCATTTT |
| | | CTCCTCCTTGTATAAATCCTGGTTGCTGTCTCTTT |
| | | ATGAGGAGTTGTGGCCCGTTGTCAGGCAACGTGGC |
| | | GTGGTGTGCACTGTGTTTGCTGACGCAACCCCCAC |
| | | TGGTTGGGGCATTGCCACCACCTGTCAGCTCCTTT |
| | | CCGGGACTTTCGCTTTCCCCCTCCCTATTGCCACG |
| | | GCGGAACTCATCGCCGCCTGCCTTGCCCGCTGCTG |
| | | GACAGGGGCTCGGCTGTTGGGCACTGACAATTCCG |
| | | TGGTGTTGTCGGGGAAATCATCGTCCTTTCCTTGG |
| | | CTGCTCGCCTGTGTTGCCACCTGGATTCTGCGCGG |
| | | GACGTCCTTCTGCTACGTCCCTTCGGCCCTCAATC |
| | | CAGCGGACCTTCCTTCCCGCGGCCTGCTGCCGGCT |
| | | CTGCGGCCTCTTCCGCGTCTTCGCCTTCGCCCTCA |
| | | GACGAGTCGGATCTCCCTTTGGGCCGCCTCCCC |
| bovine | 6 | CGCTGATCAGCCTCGACTGTGCCTTCTAGTTGCCA |
| growth | | GCCATCTGTTGTTTGCCCCTCCCCCGTGCCTTCCT |
| hormone | | TGACCCTGGAAGGTGCCACTCCCACTGTCCTTTCC |
| (bGH) | | TAATAAAATGAGGAAATTGCATCGCATTGTCTGAG |
| polyA | | TAGGTGTCATTCTATTCTGGGGGGTGGGGTGGGGC |
| tail | | AGGACAGCAAGGGGGAGGATTGGGAAGACAATAGC |
| sequence | | AGGCATGCTGGGGATGCGGTGGGCTCTATGGCTTC |
| | | TGAGGCGGAAAGAACCAGCTGGGG |
| Left ITR | 7 | CCTGCAGGCAGCTGCGCGCTCGCTCGCTCACTGAG |
| | | GCCGCCCGGGCAAAGCCCGGGCGTCGGGCGACCTT |
| | | TGGTCGCCCGGCCTCAGTGAGCGAGCGAGCGCGCA |
| | | GAGAGGGAGTGGCCAACTCCATCACTAGGGGTTCC |
| | | T |
| Right ITR | 8 | AGGAACCCCTAGTGATGGAGTTGGCCACTCCCTCT |
| | | CTGCGCGCTCGCTCGCTCACTGAGGCCGGGCGACC |
| | | AAAGGTCGCCCGACGCCCGGGCTTTGCCCGGGCGG |
| | | CCTCAGTGAGCGAGCGAGCGCGCAGCTGCCTGCAG |
| | | G |
| First | 9 | GCGGCCAATTCAGTCGATAACTATAACGGTCCTAA |
| stuffer | | GGTAGCGATTTAAATCCCGCCATGCTACTTATCTA |
| sequence | | CGTAGCCATGCTCTAGGTA |
| Second | 10 | TCTAGCGGCCGCGAATTCGATATCAAGCTT |
| stuffer | | |
| sequence | | |
| Third | 11 | GGCGGCCGCGCACCGTCGACT |
| stuffer | | |
| sequence | | |
| Fourth | 12 | CTCGACTAGAGCATGGCTACGTAGATAAGTAGCAT |
| stuffer | | GGGGGATTTAAATTAGGGATAACAGGGTAATGGCG |
| sequence | | CGGGCCGC |
| Fifth | 13 | GGCGCCTGATGCGGTATTTTCTCCTTACGCATCTG |
| stuffer | | TGCGGTATTTCACACCGCATACGTCAAAGCAACCA |
| sequence | | TAGTACGCGCCCTGTAGCGGCGCATTAAGCGCGGG |
| | | GGGTGTGGTGGTTACGCGCAGCGTGACCGCTACAC |
| | | TTGCCAGCGCCCTAGCGCCCGCTCCTTTCGCTTTC |
| | | TTCCCTTCCTTTCTCGCCACGTTCGCCGGCTTTCC |
| | | CCGTCAAGCTCTAAATCGGGGGCTCCCTTTAGGGT |
| | | TCCGATTTAGTGCTTTACGGCACCTCGACCCCAAA |
| | | AAACTTGATTTGGGTGATGGTTCACGTAGTGCAAT |
| | | TGTATCTAAGCCGTGTAATGAGAACATCCACACCT |
| | | TAGTGAATCGATGCGCCGCTTCGGAATACCGTTTT |
| | | GGCTACCTGTTACTAAGCCCATCGCGATTTTCAGG |
| | | TATCGTGCACGTAGGGTTGCACCGCACGCATGTCG |
| | | AACTGGTGGCGAAGTACGATTCCACGACCGGTGTA |
| | | CGATTCAACTACGCGGACGTGACGAGCTTCTTATA |
| | | TATGCTTCGCCCGCCGGACCGGCCTCGTGATGGGG |
| | | TAGCTGCGCATGAGCTTATGACAAATAACGAGTGT |
| | | GTACTTGTTTAATCATCTCACAGTGAAAGTCGGGA |
| | | GAATAGGAGCCGCTACACACATTTTACCGCAACTA |
| | | CACCTAACTGAGATACTACCATAGATGACTACCCA |
| | | TCCCTCTAGGCCTTAGATAACCGGATACAGTGACT |
| | | TTGATAGGTTTGTGGGGTACAGCTATGACTTGCAT |
| | | AGCTGCGTATGGAGGAAGGAACTTTTGCGTGTTAG |
| | | TATGTTGACCCGTGTATTACGCATGCGGGTAGATC |
| | | ATGTGGGTAGAGACATCCAGGTCAAGTTCTCGACC |
| | | TTCTCGTGGGAGGTGATCCAGTTCACTATAGGACC |
| | | ATTCCATTCGAGCATGGCACTAAGTACGCCGTCCC |
| | | CATTCTGGTAATCATCATCCCTATCAGGGTTTGGA |
| | | GTGAGTGGTGACGGTTATTCCCCAGGAATGGACTT |
| | | CTTATTCACAGTCGGTCACATTGGGCTACTCCTTG |
| | | GGTCTTCCGCTTGGCCCGGTCTGTTGGGCCGCCAT |
| | | TGCGTGAGTTTCGGCCCTGTGCTGCGCTGTGTCGG |
| | | CGATTCTCATTGGGGCCTCACATCTGGAAACCCCG |
| | | ACCTATTTTGACGGCACCATTGGCGGAAGTTGCTG |
| | | GGCTTGTGCACCGTGAAGTCCTCCGCGGGCCTCCC |
| | | CCCTACAAAAGACGATAAGCTCCGGCAAGCAGCAT |
| | | TGATCAACGCAAGGATCGGCGATATTAACAGAGAT |
| | | ACGGCTGATTACTCTTGTTGGTGTGGTATCGCTAA |
| | | ACTGCGCCGCGGAGCCTTATGGCAAAGTCGTCCGC |
| | | GAACCATTCCGGTAGCGCTTAAGGTCCATAGCACA |
| | | TTCATCGCATCCCGGCGTGCGTTCAATTTGACGAC |
| | | CCCTTGGCGCAAAGGTGCTGGCCACGTGCTAAATT |
| | | AAAGCGGCTGCACTGCTGTAAGGTCCGTTACGGAG |
| | | AGGGGGGCCTGGGGAGAGCACTAGCCCATCGACCT |
| | | GTACAGGAACACTCTATATTGTTCTCGGACGGACA |
| | | GATTACTAGAGTGCCGCTTTCAGCCCCCCTGTCGT |
| | | CGCCGACGTCTGTAATATGGCGTTGTTGTGATTCC |
| | | ACTCTATTGAGGCATTAACTGATGCGTAAGGAGAT |
| | | CTGGAATGAACTGGCCTATGTCACTGAAACTGTGC |
| | | AAACACCCAATGTCGTTAGTGTAGGTTCTGACCGA |
| | | TACCTGCTTCGTTGAGAACTCACAATTTTACAACT |
| | | GGGGACATAATCCCTACGCCCATCATCTACTCGCG |
| | | TCCCTGTGGCTCCAGTTCATGTAATGGGAGAGTAT |
| | | CCTCCACAAGATCTAGTGCTATGGTGGTATAGTAA |
| | | GCTCGTACTGTGGTAGACGCGACACGGGTAGGACC |
| | | ATCAGTAATAAGGATAGTGCGAAAGCTCACAGACC |
| | | ACTGCCTATAGGGGGTGCTTACTTTTACAAAAAGC |
| | | GACTGTCAGTATAACCCCACGAGGATTCGAAAAGG |
| | | TGAACCGACCCAGACGATCCGGAGGCACGGGCCTC |
| | | AAAGCCGCGACACGACGGCTGTCGGCCGGTAACAG |
| | | TAACCCCGGAGTGAACTCCTATGGGGCTGGATAGA |
| | | ACAGCCCTGGTGGGCCCCATCAGCAACCCGAATAC |
| | | GTGGCTTTTCGGGAGGCGGCCGGAGGGGCGATGTC |
| | | TTCCACTATTCGAGGCCGTTCGTTAATACTTGTTG |
| | | CGTTCCTAGCCGCTATATTTGTCTCTTTGCCGACT |
| | | AATGTGGACAAGCACACCATAGCCATTTATCGGAG |
| | | CGCCTCGGAATACGGTATGAGCAGGCGCCTCGTGA |
| | | GACCATTGCGAATACCAGGTGTCCTGTGGCGCCTG |
| | | ATGCGGTATTTTCTCCTTACGCATCTGTGCGGTAT |
| | | TTCACACCGCATACGTCAAAGCAACCATAGTACGC |
| | | GCCCTGTAGCGGCGCATTAAGCGCGGCGGGTGTGG |
| | | TGGTTACGCGCAGCGTGACCGCTACACTTGCCAGC |
| | | GCCCTAGCGCCCGCTCCTTTCGCTTTCTTCCCTTC |
| | | CTTTCTCGCCACGTTCGCCGGCTTTCCCCGTCAAG |
| | | CTCTAAATCGGGGGCTCCCTTTAGGGTTCCGATTT |
| | | AGTGCTTTACGGCACCTCGACCCCAAAAAACTTGA |
| | | TTTGGGTGATGGTTCACGTAGTGGGCCATCGCCCT |
| | | GATAGACGGTTTTTCGCCCTTTGACGTTGGAGTCC |
| | | ACGTTCTTTAATAGTGGACTCTTGTTCCAAACTGG |
| | | AACAACACTCAACCCTATCTCGGGCTATTCTTTTG |
| | | ATTTATAAGGGATTTTGCCGATTTCGGCCTATTGG |
| | | TTAAAAAATGAGCTGATTTAACAAAAATTTAACGC |
| | | GAATTTTAACAAAATATTAACGTTTACAATTTTAT |
| | | GGTGCACTCTCAGTACAATCTGCTCTGATGCCGCA |
| | | TAGTTAAGCCAGCCCCGACACCCGCCAACACCCGC |
| | | TGACGCGCCCTGACGGGCTTGTCTGCTCCCGGCAT |
| | | CCGCTTACAGACAAGCTGTGACCGTCTCCGGGAGC |
| | | TGCATGTGTCAGAGGTTTTCACCGTCATCACCGAA |
| | | ACGCGCGAGACGAAAGGGCCTCGTGATACGCCTAT |
| | | TTTTATAGGTTAATGTCATGATAATAATGGTTTCT |
| | | TAGACGTCAGGTGGCACTTTTCGGGGAAATGTGCG |
| | | CGGAACCCCTATTTGTTTATTTTTCTAAATACATT |
| | | CAAATATGTATCCGCTCATGAGACAATAACCCTGA |
| | | TAAATGCTTCAATAATATTGAAAAAGGAAGAGT |
| Sixth | 14 | TAACTGTCAGACCAAGTTTACTCATATATACTTTA |
| stuffer | | GATTGATTTAAAACTTCATTTTTAATTTAAAAGGA |
| sequence | | TCTAGGTGAAGATCCTTTTTGATAATCTCATGACC |
| | | AAAATCCCTTAACGTGAGTTTTCGTTCCACTGAGC |
| | | GTCAGACCCC |
| Seventh | 15 | CATGTACATGT |
| stuffer | | |
| sequence | | |
| Kanamycin | 16 | ATGAGCCATATTCAACGGGAAACGTCTTGCTCGAG |
| resistance | | GCCGCGATTAAATTCCAACATGGATGCTGATTTAT |
| gene | | ATGGGTATAAATGGGCTCGCGATAATGTCGGGCAA |
| | | TCAGGTGCGACAATCTATCGATTGTATGGGAAGCC |
| | | CGATGCGCCAGAGTTGTTTCTGAAACATGGCAAAG |
| | | GTAGCGTTGCCAATGATGTTACAGATGAGATGGTC |
| | | AGACTAAACTGGCTGACGGAATTTATGCCTCTTCC |
| | | GACCATCAAGCATTTTATCCGTACTCCTGATGATG |
| | | CATGGTTACTCACCACTGCGATCCCCGGGAAAACA |
| | | GCATTCCAGGTATTAGAAGAATATCCTGATTCAGG |
| | | TGAAAATATTGTTGATGCGCTGGCAGTGTTCCTGC |
| | | GCCGGTTGCATTCGATTCCTGTTTGTAATTGTCCT |
| | | TTTAACAGCGATCGCGTATTTCGTCTCGCTCAGGC |
| | | GCAATCACGAATGAATAACGGTTTGGTTGATGCGA |
| | | GTGATTTTGATGACGAGCGTAATGGCTGGCCTGTT |
| | | GAACAAGTCTGGAAAGAAATGCATAAGCTTTTGCC |
| | | ATTCTCACCGGATTCAGTCGTCACTCATGGTGATT |
| | | TCTCACTTGATAACCTTATTTTTGACGAGGGGAAA |
| | | TTAATAGGTTGTATTGATGTTGGACGAGTCGGAAT |
| | | CGCAGACCGATACCAGGATCTTGCCATCCTATGGA |
| | | ACTGCCTCGGTGAGTTTTCTCCTTCATTACAGAAA |
| | | CGGCTTTTTCAAAAATATGGTATTGATAATCCTGA |
| | | TATGAATAAATTGCAGTTTCATTTGATGCTCGATG |
| | | AGTTTTTCTAA |
| pUC origin | 17 | GTAGAAAAGATCAAAGGATCTTCTTGAGATCCTTT |
| | | TTTTCTGCGCGTAATCTGCTGCTTGCAAACAAAAA |
| | | AACCACCGCTACCAGCGGTGGTTTGTTTGCCGGAT |
| | | CAAGAGCTACCAACTCTTTTTCCGAAGGTAACTGG |
| | | CTTCAGCAGAGCGCAGATACCAAATACTGTCCTTC |
| | | TAGTGTAGCCGTAGTTAGGCCACCACTTCAAGAAC |
| | | TCTGTAGCACCGCCTACATACCTCGCTCTGCTAAT |
| | | CCTGTTACCAGTGGCTGCTGCCAGTGGCGATAAGT |
| | | CGTGTCTTACCGGGTTGGACTCAAGACGATAGTTA |
| | | CCGGATAAGGCGCAGCGGTCGGGCTGAACGGGGGG |
| | | TTCGTGCACACAGCCCAGCTTGGAGCGAACGACCT |
| | | ACACCGAACTGAGATACCTACAGCGTGAGCTATGA |
| | | GAAAGCGCCACGCTTCCCGAAGGGAGAAAGGCGGA |
| | | CAGGTATCCGGTAAGCGGCAGGGTCGGAACAGGAG |
| | | AGCGCACGAGGGAGCTTCCAGGGGGAAACGCCTGG |
| | | TATCTTTATAGTCCTGTCGGGTTTCGCCACCTCTG |
| | | ACTTGAGCGTCGATTTTTGTGATGCTCGTCAGGGG |
| | | GGCGGAGCCTATGGAAAAACGCCAGCAACGCGGCC |
| | | TTTTTACGGTTCCTGGCCTTTTGCTGGCCTTTTGC |
| | | TCA |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The invention described herein also includes the in vivo reprogramming of cells to photoreceptor cells, directly demonstrating an in vivo gene therapy application. In particular, the inventors describe use of a gene therapy approach to prevent vision loss associated with degeneration or loss of rod photoreceptor cells.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors, liposomes, and other gene delivery vehicles.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as a “rAAV vector particle” or simply a “rAAV vector”. Thus, production of rAAV particle necessarily includes production of rAAV vector, as such a vector is contained within a rAAV particle.

The term “replication defective” as used herein relative to an AAV viral vector of the invention means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

An “AAV variant” or “AAV mutant” as used herein refers to a viral particle composed of: a) a variant AAV capsid protein, where the variant AAV capsid protein comprises at least one amino acid difference (e.g., amino acid substitution, amino acid insertion, amino acid deletion) relative to a corresponding parental AAV capsid protein, and where the variant capsid protein confers increased infectivity of a retinal cell compared to the infectivity of the retinal cell by an AAV virion comprising the corresponding parental AAV capsid protein, where the AAV capsid protein does not comprise an amino acid sequence present in a naturally occurring AAV capsid protein; and b) a heterologous nucleic acid comprising a nucleotide sequence encoding a heterologous gene product.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell, e.g. a transgene as described herein. In general, the heterologous polynucleotide is flanked by at least one, and generally by two AAV inverted terminal repeat sequences (ITRs). The term rAAV vector encompasses both rAAV vector particles and rAAV vector plasmids.

As used herein, the term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, a transgene is a gene (e.g. DNA or RNA, preferably mRNA) that is delivered to a cell by a vector.

As used herein, the term “gene product” refers to the desired expression product of a polynucleotide sequence such as a polypeptide, peptide, protein or RNA.

As used herein, the terms “polypeptide,” “peptide,” and “protein” refer to polymers of amino acids of any length. The terms also encompass an amino acid polymer that has been modified; for example, disulfide bond formation, glycosylation, lipidation, phosphorylation, or conjugation with a labeling component.

As used herein, the terms “sequence identity,” e.g. “% sequence identity,” refers to the degree of identity between two or more polynucleotides when aligned using a nucleotide sequence alignment program; or between two or more polypeptide sequences when aligned using an amino acid sequence alignment program. Similarly, the term “identical” or percent “identity” when used herein in the context of two or more nucleotide or amino acid sequences refers to two sequences that are the same or have a specified percentage of amino acid residues or nucleotides when compared and aligned for maximum correspondence, for example as measured using a sequence comparison algorithm, e.g. the Smith-Waterman algorithm, etc., or by visual inspection. For example, the percent identity between two amino acid sequences may be determined using the Needleman and Wunsch, (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. As another example, the percent identity between two nucleotide sequences may be determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25:3389-3402). When utilizing BLAST and Gapped BLAST programs, the default parameters of the respective programs (e.g., XBLAST and NBLAST) can be used.

The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence.

The term “expression” as used herein encompasses the transcription and/or translation of an endogenous gene, a transgene or a coding sequence in a cell.

An “expression vector” as used herein encompasses a vector, e.g. plasmid, minicircle, viral vector, liposome, and the like as discussed above or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g. promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific (such as glial cell-specific), tissue-specific, or species specific. Promoters may “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

A “polyadenylation signal sequence” as used herein encompasses a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

As used herein, the terms “operatively linked” or “operably linked” refers to a juxtaposition of genetic elements, e.g. promoter, enhancer, termination signal sequence, polyadenylation sequence, etc., wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained. As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. As another example, a promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

The term “endogenous” as used herein with reference to a nucleotide molecule or gene product refers to a nucleic acid sequence, e.g. gene or genetic element, or gene product, e.g. RNA, protein, that is naturally occurring in or associated with a host virus or cell.

The term “native” as used herein refers to a nucleotide sequence, e.g. gene, or gene product, e.g. RNA, protein, that is present in a wildtype virus or cell.

The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the biological activity of a polypeptide or functional fragment or variant thereof refers to the ability of the polypeptide or functional fragment or variant thereof to carry out its native functions of, e.g., binding, enzymatic activity, etc. For example, a biologically active fragment or variant of a transcription factor retains the ability to bind to DNA and regulate transcription. Typically, the biologically active fragment or variant of regulates transcription to a level at least 70%, 75%, 80%, 85%, 90% or 95% of wildtype protein, preferably human.

Further, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The terms “administering” or “introducing”, as used herein refer to delivery of a vector for recombinant protein expression to a cell, to cells and/or organs of a subject, or to a subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

Typically, a cell is referred to as “transduced”, “infected”; “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

A source cell is determined to be converted to a target cell, or become a target-like cell, by a method of the invention when it displays at least one characteristic of the target cell type, i.e. a rod photoreceptor cell. For example, a human Müller glial will be identified as converted to a rod photoreceptor-like cell, when a cell displays at least one characteristic of the rod photoreceptor cell type. Typically, a cell will display 1, 2, 3, 4, 5, 6, 7, 8 or more characteristics (or markers) of a rod photoreceptor cell. For example, where the target cell is a rod photoreceptor cell, a cell is identified or determined to be a rod photoreceptor-like cell when up-regulation of any one or more photoreceptor cell markers and/or change in cell morphology is detectable, preferably, the increase in RHO mRNA expression. Other examples of photoreceptor markers include RHO, MYO7A, PDE6B, CNGB1, NR2E3, ROM1, MEF2C, ELOVL4, NRL, GNAT1, CNGA1, SAG, GNGT1, an electrophysiological response in a photopic condition, for example, as described in the Examples. Markers RHO, MYO7A, PDE6B, CNGB1, NR2E3, ROM1, MEF2C, and ELOVL4 are also markers of rod photoreceptor cells. Additional examples of photoreceptor markers include the opsins that are light-detecting molecules. For example, rhodopsin (rod photoreceptor cells), and recoverins (rod photoreceptor cells, cone photoreceptor cells). In any aspect of the invention, the target cell characteristic may be determined by analysis of cell morphology, gene expression profiles, activity assay, protein expression profile, surface marker profile, or differentiation ability. Examples of characteristics or markers include those that are described herein and those known to the skilled person.

The transcription factors referred to herein are referred to by the HUGO Gene Nomenclature Committee (HGNC) Symbol. Exemplary nucleotide sequences for each transcription factor are shown in Table 1 below. The nucleotide sequences are derived from the Ensembl database (Flicek et al. (2014). Nucleic Acids Research Volume 42, Issue D1. Pp. D749-D755) version 83. Also contemplated for use in the invention is any homolog, ortholog or paralog of a transcription factor referred to herein.

The skilled person will appreciate that this information may be used in performing the methods of the present invention, for example, for the purposes of providing increased amounts of transcription factors in source cells, or providing nucleic acids or the like for recombinantly expressing a transcription factor in a source cell.

TABLE 1
Accession numbers identifying nucleotide sequences and amino acid
sequences of transcription factors and proteins referred to herein.

| Transcription | | | |
|---|---|---|---|
| factor Associated | | | |
| Gene Name | Abbreviation | Ensembl Gene ID | Uniprot ID |
| ASCL1 | A | ENSG00000139352 | P50553 |
| NEUROD1 | N | ENSG00000162992 | Q13562 |
| NRL | Nr | ENSG00000129535 | P54845 |
| NR2E3 | Nr2 | ENSG00000278570 | Q9Y5X4 |
| RAX | R | ENSG00000134438 | Q9Y2V3 |
| RORB | Ro | ENSG00000198963 | Q92753 |
| OTX2 | O | ENSG00000165588 | P32243 |
| CRX | C | ENSG00000105392 | O43186 |
| PAX6 | P | ENSG00000007372 | P26367 |

The term a “variant refers to a polypeptide that is at least 70%, 80%, 85%, 90%, 95%, 98%, or 99% identical to the full length polypeptide. The present invention contemplates the use of variants of the transcription factors described herein, including the sequences listed in Table 1. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof has a functional activity of interest such as the ability to promote conversion of a source cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a source cell to a target cell type can be assessed using the assays as disclose herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

The term “increasing the amount of” with respect to increasing an amount of a transcription factor, refers to increasing the quantity of the transcription factor in a cell of interest (e.g., a source cell such as a fibroblast or keratinocyte cell). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a glial cell into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it).

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

A nucleic acid or vector comprising a nucleic acid as described herein may include a sequence encoding any one or more of the amino acid sequences listed in Table 1.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or source cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g. EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The terms “treatment”, “treating” and the like are used herein to generally mean obtaining a desired pharmacologic and/or physiologic effect. “Treatment” as used herein in relation includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The various compositions and methods of the invention are described below. Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### Gene Therapy Vectors

As alluded to above, in some aspects of the present invention, nucleic acids as described herein are used to deliver genes to retinal cells of an animal.

Any convenient vector, such as a gene therapy vector or gene delivery vector (used interchangeably herein), that finds use delivering nucleic acids or nucleotide sequences as described herein to cells in the retina is encompassed by the vectors of the present disclosure. For example, the vector may comprise single or double stranded nucleic acid, e.g. single stranded or double stranded DNA or RNA. For example, the gene delivery vector may be a naked DNA or RNA, e.g. a plasmid, a minicircle, etc. As another example, the gene delivery vector may be a virus, e.g. an adenovirus, an adeno-associated virus (AAV), baculovirus or a retrovirus, e.g. Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) or lentivirus. While embodiments encompassing the use of adeno-associated virus are described in greater detail below, it is expected that the ordinarily skilled artisan will appreciate that similar knowledge and skill in the art can be brought to bear on non-AAV gene therapy vectors as well. See, for example, the discussion of retroviral vectors in, e.g., U.S. Pat. Nos. 7,585,676 and 8,900,858, and the discussion of adenoviral vectors in, e.g. U.S. Pat. No. 7,858,367, the full disclosures of which are incorporated herein by reference.

Gene therapy vectors, e.g., lentivirus and baculovirus, virions encapsulating the polynucleotide cassettes of the present disclosure, may be produced using standard methodology. In some embodiments, the gene delivery vector is a recombinant adeno-associated virus (rAAV). In such embodiments, the expression construct encoding a set of transcription factors descried herein at (a) to (jj), or biologically active fragments or variants thereof is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the invention need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAV5, AAV6, AAV7, AAV8, AAV9, AAV10, ShH10 and ShH10Y. Preferred AAV vectors have the wild type REP and CAP genes deleted in whole or part, but retain functional flanking ITR sequences.

In such embodiments, the nucleic acid comprising an expression construct is encapsidated within an AAV capsid, which may be derived from any adeno-associated virus serotype, including without limitation, AAV1, AAV2, AAV3, AAV4, AAV5, AAV6, AAV7, AAV8, AAV9, AAV10, etc. For example, the AAV capsid may be a wild type, or native, capsid. Wild type AAV capsids of particular interest include AAV2, AAV5, AAV6 and AAV9. However, as with the ITRs, the capsid need not have a wild-type nucleotide sequence, but rather may be altered by the insertion, deletion or substitution of nucleotides in the VP1, VP2 or VP3 sequence, so long as the capsid is able to transduce rod cells. Put another way, the AAV capsid may be a variant AAV capsid. Variant AAV capsids of particular interest include those comprising a peptide insertion within residues 580-600 of AAV2 or the corresponding residues in another AAV, e.g. LGETTRP, NETITRP, KAGQANN, KDPKTTN, KDTDTTR, RAGGSVG, AVDTTKF, or STGKVPN, as disclosed in US Application No. US 2014/0294771, the full disclosure of which is incorporated by reference herein. In some embodiments, the AAV vector is a “pseudotyped” AAV created by using the capsid (cap) gene of one AAV and the rep gene and ITRs from a different AAV, e.g. a pseudotyped AAV2 created by using rep from AAV2 and cap from AAV1, AAV3, AAV4, AAV5, AAV6, AAV7, AAV8, or AAV9 together with a plasmid containing a vector based on AAV2. For example, the AAV vector may be rAAV2/1, rAAV2/3, rAAV2/4, rAAV2/5, rAAV2/6, rAAV2/7, rAAV2/8, rAAV2/9, etc. Preferably, the rAAV is replication defective, in that the AAV vector cannot independently further replicate and package its genome. For example, when rod cells are transduced with rAAV virions, the gene is expressed in the transduced rod cells, however, due to the fact that the transduced rod cells lack AAV rep and cap genes and accessory function genes, the rAAV is not able to replicate.

In the case of rAAV virions, an AAV expression vector according to the invention may be introduced into a producer cell, followed by introduction of an AAV helper construct, where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV.

In preparing the rAAV compositions, any host cells for producing rAAV virions may be employed, including, for example, mammalian cells (e.g. 293 cells), insect cells (e.g. SF9 cells), microorganisms and yeast. Host cells can also be packaging cells in which the AAV rep and cap genes are stably maintained in the host cell or producer cells in which the AAV vector genome is stably maintained and packaged. Exemplary packaging and producer cells are derived from SF-9, 293, A549 or Hela cells. AAV vectors are purified and formulated using standard techniques known in the art. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety.

Any suitable method for producing viral particles for delivery of the nucleic acids (e.g. DNA or RNA) or nucleotide sequences as described herein can be used, including but not limited to those described in the examples that follow. Any concentration of viral particles suitable to effectively transduce retinal cells can be prepared for contacting those cells in vitro or in vivo. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL. Similarly, any total number of viral particles suitable to provide appropriate transduction of retinal cells to confer the desired effect or treat the disease can be administered to the mammal or to the primate's eye. In various preferred embodiments, at least 10⁸; 5×10⁸; 10⁹; 5×10⁹; 10¹⁰, 5×10¹⁰, 10¹¹; 5×10¹¹; 10¹²; 10¹³; 5×10¹²; 10¹³; 1.5×10¹³; 3×10¹³; 5×10¹³; 7.5×10¹³; 9×10¹³; 1×10¹⁴ viral particles, or 5×10¹⁴ viral particles or more, but typically not more than 1×10¹⁵ viral particles are injected per eye. Any suitable number of administrations of the vector to the subject eye can be made. In one embodiment, the methods comprise a single administration; in other embodiments, multiple administrations are made over time as deemed appropriate by an attending clinician.

The vector may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some cases, the unit dosage is at most about 5×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴1×10¹⁵, 1×10¹⁶, 1×10¹⁷, and 1×10¹⁸ MOI.

In some aspects, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

### Formulation

The nucleic acid or vector according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, liposome suspension or any other suitable form that may be administered to a person or animal in need of treatment. However, preferably the construct or vector is formulated for suitable administration to the subject's eye, preferably suitable for retinal administration, preferably by injection, more preferably by retinal injection (e.g. subretinal injection) or most preferably by intravitreal injection. It will be appreciated that the vehicle of medicaments according to the invention should be one which is well-tolerated by the subject to whom it is given.

It will be appreciated that the amount of the nucleic acid or vector that is required is determined by its biological activity and bioavailability, which in turn depends on the mode of administration, the physiochemical properties of the nucleic acid or vector and whether it is being used as a monotherapy or in a combined therapy. The frequency of administration will also be influenced by the half-life of the construct or vector within the subject being treated. Optimal dosages to be administered may be determined by those skilled in the art, and will vary with the particular nucleic acid or vector in use, the strength of the pharmaceutical composition, the mode of administration, and the advancement of the retinal disorder. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the nucleic acid or vector according to the invention may be used for treating, ameliorating, or preventing a retinal disorder, depending upon the nucleic acid or vector used.

The nucleic acid or vector may be administered before, during or after onset of the retinal disorder. Daily doses may be given as a single administration (e.g. a single daily injection). Alternatively, the nucleic acid or vector may require administration twice or more times during a day. As an example, the nucleic acid or vector may be administered as two (or more depending upon the severity of the retinal disorder being treated) daily doses of between 0.07 μg and 700 mg (i.e. assuming a body weight of 70 kg). A patient receiving treatment may take a first dose upon waking and then a second dose in the evening (if on a two-dose regime) or at 3- or 4-hourly intervals thereafter. Alternatively, a slow release device may be used to provide optimal doses of the nucleic acid or vector according to the invention to a patient without the need to administer repeated doses.

However, the pharmaceutical vehicle may be a liquid, and the pharmaceutical composition is in the form of a solution. Liquid vehicles are used in preparing solutions, suspensions, emulsions, syrups, elixirs and pressurized compositions. The nucleic acid or vector according to the invention may be dissolved or suspended in a pharmaceutically acceptable liquid vehicle such as water, an organic solvent, a mixture of both or pharmaceutically acceptable oils or fats.

Liquid pharmaceutical compositions, which are sterile solutions or suspensions, can be utilized by, for example, intraocular, particularly intravitreal or subretinal injection. The nucleic acid or vector may be prepared as a sterile solid composition that may be dissolved or suspended at the time of administration using sterile water, saline, or other appropriate sterile injectable medium.

For instances in which retinal glial cells are to be contacted in vivo, an nucleic acid of the invention as described herein (e.g. synthetic mRNA), or a vector, preferably an AAV vector, of the invention as described herein, or a recombinant AAV of the invention as described herein can be treated as appropriate for delivery to the eye. In particular, the present invention includes pharmaceutical compositions comprising an nucleic acid of the invention as described herein, a vector, preferably an AAV vector of the invention as described herein, or a recombinant AAV of the invention as described herein and a pharmaceutically-acceptable carrier, diluent or excipient. The nucleic acid of the invention as described herein, vector, preferably an AAV vector of the invention as described herein, or recombinant AAV of the invention as described herein can be combined with pharmaceutically-acceptable carriers, diluents and reagents useful in preparing a formulation that is generally safe, non-toxic, and desirable, and includes excipients that are acceptable for primate use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide.

Pharmaceutical compositions suitable for internal use in the present invention further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid to the extent that easy syringability exists. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the nucleic acid of the invention as described herein, vector, preferably an AAV vector, of the invention as described herein, or recombinant AAV of the invention as described herein in the required amount in an appropriate solvent with one or a combination of ingredients enumerated above, as required, followed by filtered sterilization. Generally, dispersions are prepared by incorporating the nucleic acid of the invention as described herein, vector, preferably AAV vector, of the invention as described herein, or recombinant AAV of the invention as described herein into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bio-equivalents.

The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see Handbook of Pharmaceutical Salts: Properties, Selection, and Use by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The nucleic acid of the invention as described herein, vector preferably AAV vector of the invention as described herein, or recombinant AAV of the invention as described herein, can be incorporated into pharmaceutical compositions for administration to mammalian patients, particularly primates and more particularly humans. The subject nucleic acid of the invention as described herein, vector, preferably AAV vector, of the invention as described herein, or recombinant AAV of the invention as described herein can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the PHGDH or a biologically active fragment or variant thereof dissolved in an aqueous buffer having an acceptable pH upon reconstitution.

In some embodiments, the pharmaceutical composition provided herein comprise a therapeutically effective amount of a vector or virion in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

### Promoters

Any promoter sequences that allow expression in glial cells (preferably retinal glial cells), which is the target tissue/cell type, are useful in the present invention. These include ubiquitous promoters e.g. CAG promoter, and glial cell-specific promoters. The CAG promoter preferably comprises the cytomegalovirus (CMV) early enhancer element, the promoter, the first exon and the first intron of chicken beta-actin (CBA) gene and the splice acceptor of the rabbit beta-globin gene. Examples of glial cell-specific promoters would include, but not be limited to, the promoters for GFAP, GLAST and RLBP1.

Viral-mediated expression assays of PHGDH include Western immunoblot and immunohistochemistry analysis of treated cells.

### Methods of Treatment

In another aspect, the present invention provides a method of decreasing progression of or ameliorating vision loss associated with or cause by degeneration, or loss, of rod photoreceptor cells in a subject, the method comprising administering to the subject an nucleic acid of the invention as described herein, a vector, preferably an AAV vector, of the invention as described herein, a recombinant AAV of the invention as described herein, or a pharmaceutical composition of the invention as described herein, thereby of decreasing progression of or ameliorating vision loss associated with or cause by degeneration, or loss, of rod photoreceptor cells.

In another aspect, the present invention provides use of an nucleic acid of the invention as described herein, a vector, preferably an AAV vector, of the invention as described herein, a recombinant AAV of the invention as described herein, or a pharmaceutical composition of the invention as described herein, in the manufacture of a medicament for decreasing progression of or ameliorating vision loss associated with or cause by degeneration, or loss, of rod photoreceptor cells in a subject.

In another aspect, the present invention provides an nucleic acid of the invention as described herein, a vector, preferably an AAV vector, of the invention as described herein, a recombinant AAV of the invention as described herein, or a pharmaceutical composition of the invention as described herein, for use in decreasing progression of or ameliorating vision associated with or cause by degeneration, or loss, of rod photoreceptor cells in a subject.

In any aspect, preferably the subject is a human.

In any aspect or embodiment, the condition associated with or cause by degeneration, or loss, of rod photoreceptor cells may also be referred to as a rod cell disorder. The degeneration, or loss, of rod photoreceptor cells is associated with or causes changes in vision, typically a reduction in vision.

In some embodiments, the rod cell disorder is a retinal degenerative disorder. In some embodiments, the rod cell disorder is a macular dystrophy or retinal dystrophy. The macular dystrophy may be selected from the group consisting of Stargardt's macular dystrophy, rod dystrophy (including rod-cone dystrophy and cone-rod dystrophy), Spinocerebellar ataxia type 7, and Bardet-Biedl syndrome-1. Preferably, the macular dystrophy is Stargardt's macular dystrophy or rod-cone dystrophy. In some embodiments, the rod cell disorder is a vision disorder of the central macula or a retinal dystrophy. In certain embodiments, vision disorder of the central macula or retinal dystrophy is selected from the group consisting of age-related macular degeneration, macular telangiectasia, retinitis pigmentosa, diabetic retinopathy, retinal vein occlusions, glaucoma, choroideremia, Sorsby's fundus dystrophy, adult vitelliform macular dystrophy, Best's disease, Leber's congenital amaurosis, and X-linked retinoschisis. Preferably, the vision disorder is retinitis pigmentosa, age-related macular degeneration or diabetic retinopathy.

In any embodiment, the subject has been diagnosed with a condition associated with or cause by degeneration, or loss, of rod photoreceptor cells as described herein. Preferably, the individual has been diagnosed with a rod dystrophy. The individual may have been diagnosed with progressive rod dystrophy or stationary rod dystrophy. The rod dystrophy may be a rod-cone dystrophy or a cone-rod dystrophy.

In some such embodiments, the method further comprises detecting a change in the condition or disorder symptoms. Including any symptom described herein. In some such embodiments, the change comprises a stabilization in the health of the existing or reprogrammed rod cells and/or a reduction in the rate of visual acuity loss of the subject. In certain such embodiments, the change comprises an improvement in in the visual acuity of the subject.

In some such embodiments, the method further comprises detecting a change in the condition or disorder symptoms, wherein the change comprises an increase in the vision of the subject.

In any aspect of the present invention, a nucleic acid of the invention as described herein, a vector of the invention as described herein, an AAV vector of the invention as described herein, a recombinant AAV of the invention as described herein, or a pharmaceutical composition of the invention as described herein, is administered to the subject by retinal administration, preferably by retinal injection (e.g. subretinal or intravitreal injection) into an affected eye of said subject.

In another aspect, the present invention provides for a composition comprising any of the vectors (e.g. AAV vectors) of the invention as described herein or rAAV of the invention as disclosed herein and a pharmaceutically acceptable carrier, excipient or diluent.

In some embodiments, the loss of photoreceptors is a complete loss of rod photoreceptors. In some embodiments, the patient has eyesight of 20/60 or worse including 20/80 or worse, 20/100 or worse, 20/120 or worse, 20/140 or worse, 20/160 or worse, 20/180 or worse, 20/200 or worse, 20/400 or worse, 20/800 or worse, or 20/1000 or worse.

For instances in which cells are to be contacted in vivo with a subject nucleic acid or gene delivery vector as described herein, the subject may be any mammal, e.g. rodent (e.g. mice, rats, gerbils), rabbit, feline, canine, goat, ovine, pig, equine, bovine, or primate.

The methods and compositions of the present disclosure find use in the treatment of any condition that can be addressed, at least in part, by producing functional rod photoreceptor cells. Thus, the compositions and methods of the present disclosure find use in the treatment of individuals in need of a rod cell therapy. By a person in need of a rod cell therapy, it is meant an individual having or at risk of developing a rod cell disorder. By a “rod cell disorder” it is meant any disorder impacting retinal rod cells, including but not limited to vision disorders of the eye that are associated with a defect within rod cells, i.e. a rod-instrinsic defect, e.g. macular dystrophies such as Stargardt's macular dystrophy, rod dystrophy, rod-cone dystrophy, Spinocerebellar ataxia type 7, and Bardet-Biedl syndrome-1; as well as vision disorders of the central macula (within primates) that may be treated by targeting rod cells, e.g. age-related macular degeneration, macular telangiectasia, retinitis pigmentosa, diabetic retinopathy, retinal vein occlusions, glaucoma, Sorsby's fundus dystrophy, adult vitelliform macular dystrophy, Best's disease, rod-cone dystrophy, Leber's congenital amaurosis, and X-linked retinoschisis.

Stargardt's macular dystrophy. Stargardt's macular dystrophy, also known as Stargardt Disease and fundus flavimaculatus, is an inherited form of juvenile macular degeneration that causes progressive vision loss usually to the point of legal blindness. The onset of symptoms usually appears between the ages of six and thirty years old (average of about 16-18 years). Mutations in several genes, including ABCA4, CNGB3, ELOVL4, PROM1, are associated with the disorder. Symptoms typically develop by twenty years of age, and include wavy vision, blind spots, blurriness, impaired color vision, and difficulty adapting to dim lighting. The main symptom of Stargardt disease is loss of visual acuity, which ranges from 20/50 to 20/200. In addition, those with Stargardt disease are sensitive to glare; overcast days offer some relief. Vision is most noticeably impaired when the macula is damaged, which can be observed by fundus exam.

Age-related macular degeneration. Age-related macular degeneration (AMD) is one of the leading causes of vision loss in people over the age of 50 years. AMD mainly affects central vision, which is needed for detailed tasks such as reading, driving, and recognizing faces. The vision loss in this condition results from a gradual deterioration of photoreceptors in the macula. Side (peripheral) vision and night vision are generally not affected.

Researchers have described two major types of age-related macular degeneration, known as the dry, or “nonexudative” form, and the wet, or “exudative” or “neovascular”, form, both of which may be treated by delivering transgenes in the context of the subject polynucleotide cassettes.

Dry AMD is characterized by a buildup of yellow deposits called drusen between the retinal pigment epithelium and the underlying choroid of the macula, which may be observed by Fundus photography. This results in a slowly progressive loss of vision. The condition typically affects vision in both eyes, although vision loss often occurs in one eye before the other. Other changes may include pigment changes and RPE atrophy. For example, in certain cases called central geographic atrophy, or “GA”, atrophy of the retinal pigment epithelial and subsequent loss of photoreceptors in the central part of the eye is observed. Dry AMD has been associated with mutations in CD59 and genes in the complement cascade.

Wet AMD is a progressed state of dry AMD, and occurs in about 10% of dry AMD patients. Pathological changes include retinal pigment epithelial cells (RPE) dysfunction, fluid collecting under the RPE, and choroidal neovascularization (CNV) in the macular area. Fluid leakage, RPE or neural retinal detachment and bleeding from ruptured blood vessels can occur in severe cases. Symptoms of wet AMD may include visual distortions, such as straight lines appearing wavy or crooked, a doorway or street sign looking lopsided, or objects appearing smaller or farther away than they really are; decreased central vision; decreased intensity or brightness of colors; and well-defined blurry spot or blind spot in the field of vision. Onset may be abrupt and worsen rapidly. Diagnosis may include the use of an Amsler grid to test for defects in the subject's central vision (macular degeneration may cause the straight lines in the grid to appear faded, broken or distorted), fluorescein angiogram to observe blood vessel or retinal abnormalities, and optical coherence tomography to detect retina swelling or leaking blood vessels. A number of cellular factors have been implicated in the generation of CNV, among which are vascular endothelial growth factor (VEGF), platelet-derived growth factor (PDGF), pigment epithelium-derived factor (PEDF), hypoxia inducible factor (HIF), angiopoietin (Ang), and other cytokines, mitogen-activated protein kinases (MAPK) and others.

Retinitis pigmentosa. Retinitis Pigmentosa (RP) is a group of inherited disorders characterized by progressive peripheral vision loss and night vision difficulties (nyctalopia) that can lead to central vision loss. Presenting signs and symptoms of RP vary, but the classic ones include nyctalopia (night blindness, most commonly the earliest symptom in RP); visual loss (usually peripheral, but in advanced cases, central visual loss); and photopsia (seeing flashes of light). Because RP is a collection of many inherited diseases, significant variability exists in the physical findings. Ocular examination involves assessment of visual acuity and pupillary reaction, as well as anterior segment, retinal, and funduscopic evaluation. In some instances, the RP is one aspect of a syndrome, e.g. syndromes that are also associated with hearing loss (Usher syndrome, Waardenburg syndrome, Alport syndrome, Refsum disease); Kearns-Sayre syndrome (external ophthalmoplegia, lid ptosis, heart block, and pigmentary retinopathy); Abetalipoproteinemia (Fat malabsorption, fat-soluble vitamin deficiencies, spinocerebellar degeneration, and pigmentary retinal degeneration);

mucopolysaccharidoses (e.g., Hurler syndrome, Scheie syndrome, Sanfilippo syndrome); Bardet-Biedl syndrome (Polydactyly, truncal obesity, kidney dysfunction, short stature, and pigmentary retinopathy); and neuronal ceroid lipofuscinosis (Dementia, seizures, and pigmentary retinopathy; infantile form is known as Jansky-Bielschowsky disease, juvenile form is Vogt-Spielmeyer-Batten disease, and adult form is Kufs syndrome). Retinitis pigmentosa is most commonly associated with mutations in the RHO, RP2, RPGR, RPGRIP1, PDE6A, PDE6B, MERTK, PRPH2, CNGB1, USH2A, ABCA4, BBS genes.

Diabetic retinopathy. Diabetic retinopathy (DR) is damage to the retina caused by complications of diabetes, which can eventually lead to blindness. Without wishing to be bound by theory, it is believed that hyperglycemia-induced intramural pericyte death and thickening of the basement membrane lead to incompetence of the vascular walls. These damages change the formation of the blood-retinal barrier and also make the retinal blood vessels become more permeable.

There are two stages of diabetic retinopathy: non-proliferative diabetic retinopathy (NPDR), and proliferative diabetic retinopathy (PDR). Nonproliferative diabetic retinopathy is the first stage of diabetic retinopathy, and is diagnosed by fundoscopic exam and coexistent diabetes. In cases of reduced vision, fluorescein angiography may be done to visualize the vessles in the back of the eye to and any retinal ischemia that may be present. All people with diabetes are at risk for developing NPDR, and as such, would be candidates for prophylactic treatment with the subject vectors. Proliferative diabetic retinopathy is the second stage of diabetic retinopathy, characterized by neovascularization of the retina, vitreous hemorrhage, and blurred vision. In some instances, fibrovascular proliferation causes tractional retinal detachment. In some instances, the vessels can also grow into the angle of the anterior chamber of the eye and cause neovascular glaucoma. Individuals with NPDR are at increased risk for developing PDR, and as such, would be candidates for prophylactic treatment with the subject vectors.

In some embodiments, the loss of photoreceptors is a complete loss of photoreceptors. In some embodiments, the patient has eyesight of 20/60 or worse including 20/80 or worse, 20/100 or worse, 20/120 or worse, 20/140 or worse, 20/160 or worse, 20/180 or worse, 20/200 or worse, 20/400 or worse, 20/800 or worse, or 20/1000 or worse.

### Administration

In practicing the in vivo methods, a composition for in vivo reprogramming is typically delivered to the retina of the subject in an amount that is effective to result in the expression of, for example, the transgene(s) in the retinal glial cells. In some embodiments, the method comprises the step of detecting the expression of the transgene in cells of the retina, for example retinal glial cells.

In a preferred embodiment, nucleic acids, vectors, AAVs, medicaments according to the invention may be administered to a subject by injection into the blood stream, a nerve or directly into a site requiring treatment, i.e. the eye. For example, the medicament may be injected at least adjacent the retina. Injections may be intravenous (bolus or infusion) or subcutaneous (bolus or infusion), or intradermal (bolus or infusion), or intravitreal (bolus or infusion), or subretinal (bolus or infusion).

Preferably, the nucleic acid of the invention as described herein, vector, preferably AAV vector of the invention as described herein, or recombinant AAV of the invention as described herein is administered directly to the subject's eye (e.g. to the retina), preferably by injection, more preferably by retinal injection (e.g. subretinal injection) most preferably by intravitreal injection.

The composition may be administered to the retina of the by any suitable method. For example, the composition may be administered intraocularly via intravitreal injection or subretinal injection. The general methods for delivering a nucleic acid or vector via intravitreal injection or via subretinal injection may be illustrated by the following brief outlines. These examples are merely meant to illustrate certain features of the methods, and are in no way meant to be limiting.

For subretinal administration, the nucleic acid or vector can be delivered in the form of a suspension injected subretinally under direct observation using an operating microscope. Typically, a volume of 1 to 200 uL, e.g. 50 uL, 100 uL, 150 ul, or 200 uL, but usually no more than 200 uL, of the subject composition will be administered by such methods. This procedure may involve vitrectomy followed by injection of vector suspension using a fine cannula through one or more small retinotomies into the subretinal space. Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of the nucleic acid or vector, and (3) its controlled removal reduces the possibility of retinal tears and unplanned retinal detachment.

For intravitreal administration, the nucleic acid or vector can be delivered in the form of a suspension. Initially, topical anesthetic is applied to the surface of the eye followed by a topical antiseptic solution. The eye is held open, with or without instrumentation, and the nucleic acid or vector is injected through the sclera with a short, narrow, for example a 30 gauge needle, into the vitreous cavity of the eye of a subject under direct observation. Typically, a volume of 1 to 100 uL, e.g. 25 μL, 50 uL, or 100 uL, and usually no more than 100 uL, of the subject composition may be delivered to the eye by intravitreal injection without removing the vitreous. Alternatively, a vitrectomy may be performed, and the entire volume of vitreous gel is replaced by an infusion of the subject composition. In such cases, up to about 4 mL of the subject composition may be delivered, e.g. to a human eye. Intravitreal administration is generally well tolerated. At the conclusion of the procedure, there is sometimes mild redness at the injection site. There is occasional tenderness, but most patients do not report any pain. No eye patch or eye shield is necessary after this procedure, and activities are not restricted. Sometimes, an antibiotic eye drop is prescribed for several days to help prevent infection.

In practicing the methods, the composition is typically delivered to the retina of the subject in an amount that is effective to result in the expression of the transgene(s) in the rod cells. In some embodiments, the method comprises the step of detecting the expression of the transgene(s) in the cells of the retina.

There are a number of ways to detect the expression of a transgene, any of which may be used in the subject embodiments. For example, expression may be detected directly, i.e. by measuring the amount of gene product, for example, at the RNA level, e.g. by RT-PCR, Northern blot, RNAse protection; or at the protein level, e.g. by Western blot, ELISA, immunohistochemistry, and the like. As another example, expression may be detected indirectly, i.e. by detecting the impact of the gene product on the viability or function of the rod photoreceptor in the subject. For example, if the gene product encoded by the transgene improves the viability of the rod cell, the expression of the transgene may be detected by detecting an improvement in viability of the rod cell, e.g. by fundus photography, Optical coherence tomography (OCT), Adaptive Optics (AO), and the like. If the gene product encoded by the transgene alters the activity of the rod cell, the expression of the transgene may be detected by detecting a change in the activity of the rod cell, e.g. by electroretinogram (ERG) and color ERG (cERG); functional adaptive optics; colour vision tests such as pseudoisochromatic plates (Ishihara plates, Hardy-Rand-Ritter polychromatic plates), the Farnsworth-Munsell 100 hue test, the Farnsworth's panel D-15, the City university test, Kollner's rule, and the like; and visual acuity tests such as the ETDRS letters test, Snellen visual acuity test, visual field test, contrast sensitivity test, and the like, as a way of detecting the presence of the delivered polynucleotide. In some instances, both an improvement in viability and a modification in rod cell function may be detected.

In some embodiments, the method results in a therapeutic benefit, e.g. preventing the development of a disorder, halting the progression of a disorder, reversing the progression of a disorder, etc. In some embodiments, the method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy. For example, therapeutic efficacy in treating photoreceptor degeneration may be observed as a reduction in the rate of photoreceptor degeneration or a cessation of the progression of photoreceptor degeneration, effects which may be observed by, e.g., fundus photography, OCT, or AO, by comparing test results after administration of the composition to test results before administration of the subject composition. As another example, therapeutic efficacy in treating a progressive rod dysfunction may be observed as a reduction in the rate of progression of rod dysfunction, as a cessation in the progression of rod dysfunction, or as an improvement in rod function, effects which may be observed by, e.g., ERG and/or cERG; colour vision tests; functional adaptive optics; and/or visual acuity tests, for example, by comparing test results after administration of the composition to test results before administration of the subject composition and detecting a change in rod viability and/or function.

Individual doses are typically not less than an amount required to produce a measurable effect on the subject, and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount, which can be adjusted for subretinal (applied directly to where action is desired for mainly a local effect), intravitreal (applied to the vitreaous for a pan-retinal effect), or parenteral (applied by systemic routes, e.g. intravenous, intramuscular, etc.) applications. Effective amounts of dose and/or dose regimen can readily be determined empirically from preclinical assays, from safety and escalation and dose range trials, individual clinician-patient relationships.

### EXAMPLES

Here the inventors describe methods for in vivo reprogramming of photoreceptor cells through in vivo delivery of one or more expression constructs to retinal cells with a cocktail of transcription factors.

The identification of transcriptome factors is performed using an optimized CRISPRa system in mammalian cells as previously described (Fang et al. Molecular therapy. Nucleic Acids, 20 Nov. 2018, 14:184-191), which activates efficient expression of up to 9 genes simultaneously. This platform can greatly enhance the capacity to perform in vitro screening in human Müller glial cell line (MIO-M1) and may be used to identify various transcription factors or combinations that promote in vivo reprogramming into induced photoreceptors (iPH). Validation of the iPH quality may be performed by photoreceptor marker analysis using qPCR and immunocytochemistry, as well as single cell transcriptomic using the 10× Chromium system and next generation sequencing.

### Example 1—Processes for Identification and Characterisation of Transcriptome Factors

The loss of photoreceptors is a key hallmark of many incurable blinding diseases and regenerative medicine has great potentials of alleviating blindness in patients. Here the inventors describe a method for the identification of transcription factors that promote in vivo reprograming of photoreceptors (termed induced photoreceptors, iPH).

The method may adapt the CRISPR activation (CRISPRa) system to activate expression of endogenous genes, which allows for the activation of up to 9 transcription factors simultaneously (for example, the individual transcription factors or transcription factor sets, including but not limited to those shown in Table 3). This CRISPRa platform, allows for screening and identification of individual transcription factors and transcription factor combinations or cocktails that promote reprogramming of human Müller glia into iPH in vitro and may be used to promote in vivo reprogramming into iPH.

The method may use a transgene delivery system to drive expression of endogenous or exogenous genes. A transgene delivery system may encode one or more sets of transcription factors, for example, the individual transcription factors or transcription factor sets, including but not limited to those shown in Table 3. The transgene delivery system, allows for screening and identification of individual transcription factors and transcription factor combinations or cocktails that promote reprogramming of human Müller glia into iPH in vitro and may be used to promote in vivo reprogramming into iPH. One skilled in the art would recognise many methods and materials that are suitable to perform in vivo transgene delivery, including those that are described herein. Preferably, the transgene(s) are delivered using an AAV vector (particularly ShH10Y), lentiviral vector, baculoviral vector or a synthetic mRNA.

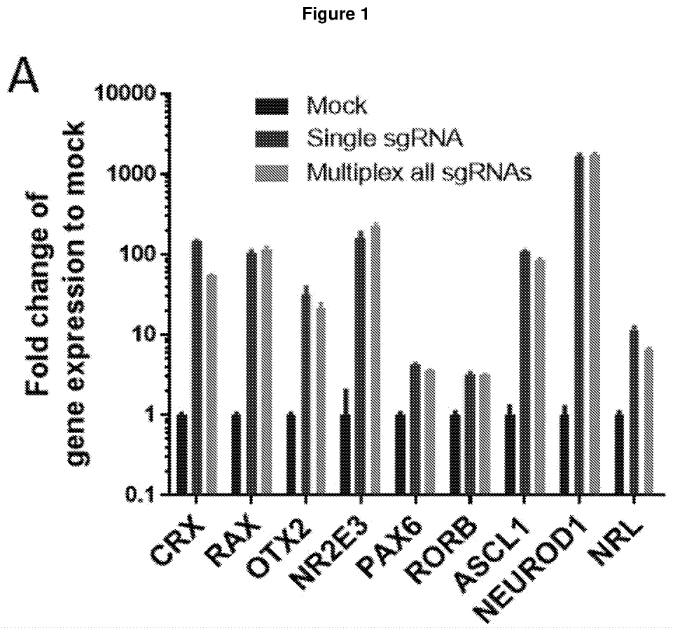

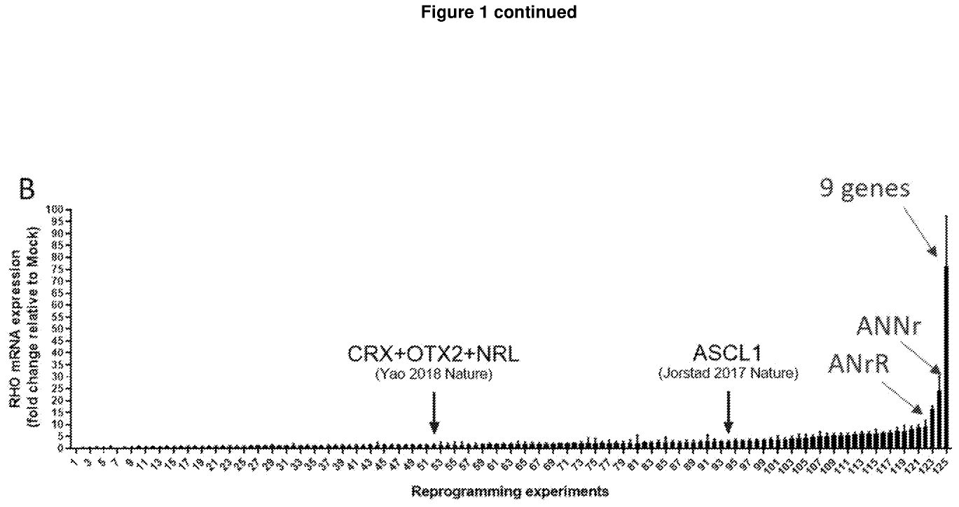

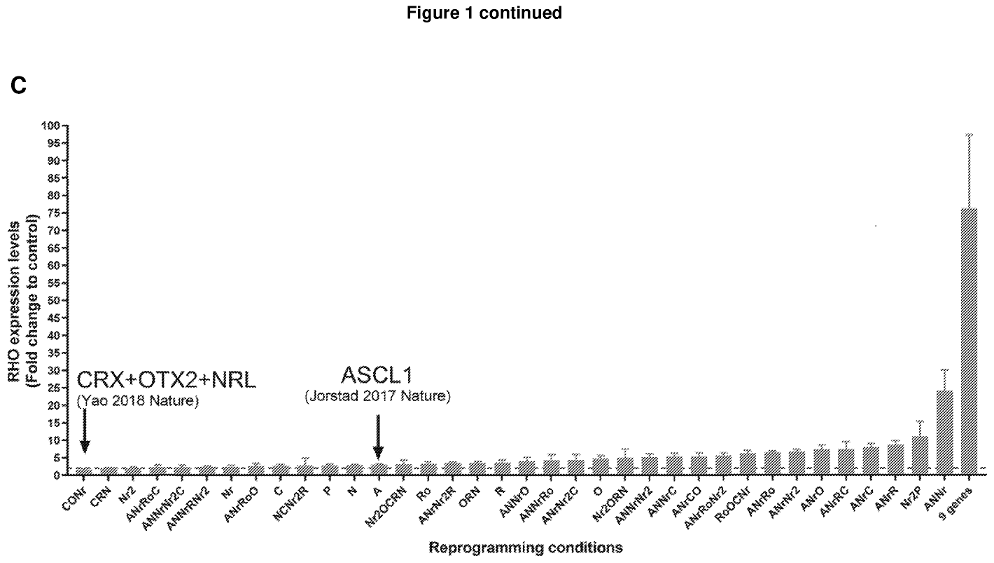

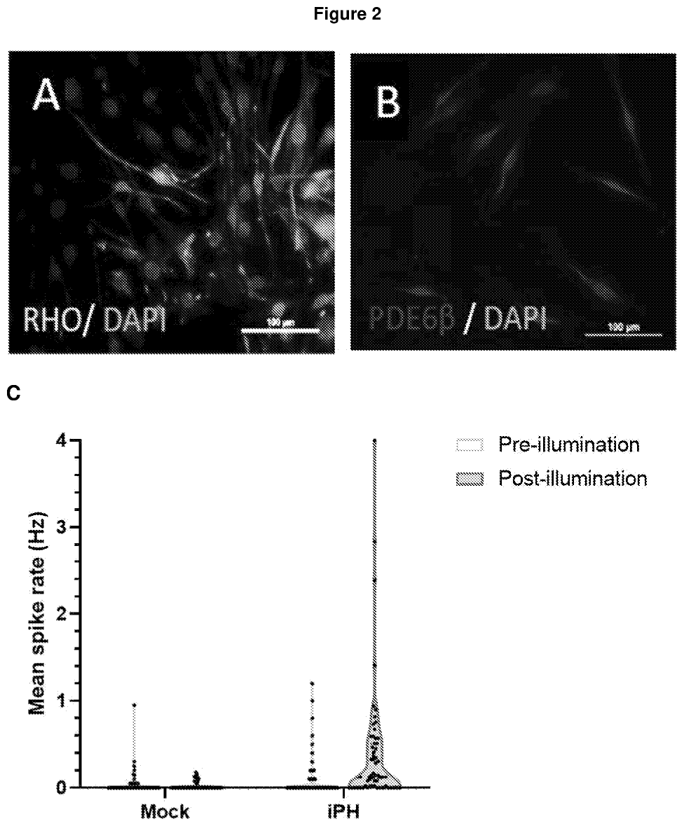

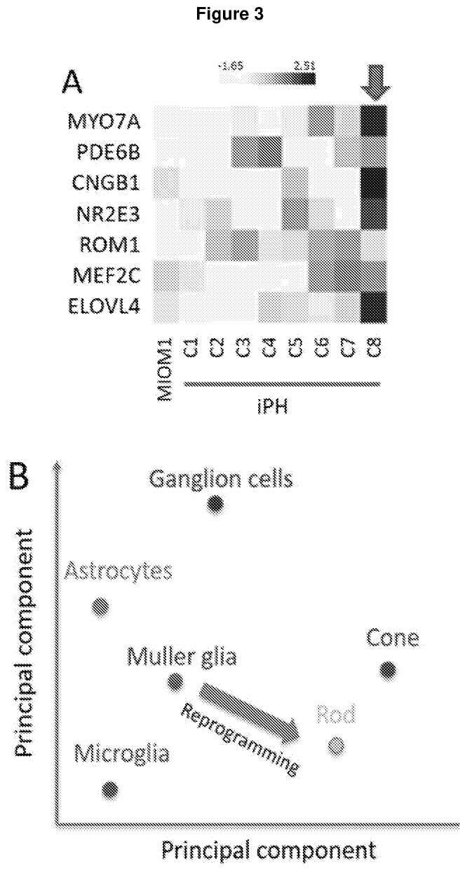

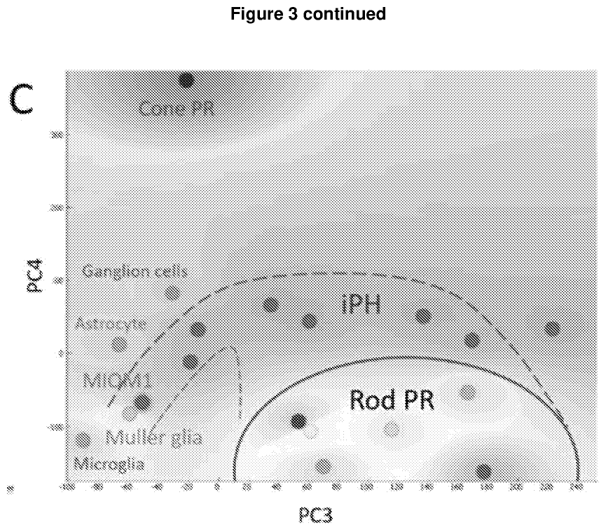

qPCR and immunocytochemical analysis may also be used to validate that the transcription factors can induce reprogramming by demonstrating that the in vitro reprogrammed iPH express a panel of photoreceptor markers, including RHO and PDE6B (See FIG. 1 and FIG. 2A-B for example). A multi-electrode array analysis, may also validate that the iPH possess functional electrophysiology (See FIG. 2C for example). To comprehensively analyse the iPH, a single cell transcriptome profiling of iPH cells may be performed. Results of a transcriptome analysis can be used to demonstrate the transition of glial to neuron through reprogramming, the activation of photoreceptor markers in iPH and the presence of different reprogramming stages (See FIG. 3 for example). Using single cell transcriptomics, the iPH can also be benchmarked against a human adult retina gene atlas. The analysis of these results can be used to show that iPH reprogramming promoted transcriptome transitions from Müller glia to photoreceptors, supporting use of the transcription factors for photoreceptor reprogramming.

The methods described herein can also be used for direct in vivo reprogramming to convert retinal cells into rod photoreceptors using the identified and validated transcriptome factors of above, providing a potential regenerative approach to the retina.

It will be understood that the methods described herein, and more particularly below, are representative examples of the processes and results that may be obtained. The present invention, includes but is not limited to any specific examples, experimental data, Figures or tables are described herein.

### Example 2—Materials and Methods

### Reprograming to Generate iPH Cells

The CRISPRa system was utilised to promote reprogramming of human MG cells to iPH cells, either by Lipofectamine transfection or lentiviral transduction. For Lipofectamine transfection, sgRNA expression cassettes were generated as described by Fang et al. supra. Human MG cells (MIO-M1) cultured in DMEM Cell Media (Thermo Fisher Scientific, cat #11995-073) containing 10% fetal bovine serum (Thermo Fisher Scientific, cat #26140079), 2 mM glutamax (Thermo Fisher Scientific, cat #35050061) and 0.5% Peniccilin-Streptomycin (Thermo Fisher Scientific, cat #15140122) were transfected with 40 ng sgRNA expression cassette and 800 ng Sp-dCas9VPR plasmid (Addgene) per well in 12 well plate format using Lipofectamine 3000 following manufacturer's instructions. From day 3 onwards, the media was switched to the Photoreceptor Cell Media, comprising Neurobasal medium (Thermo Fisher Scientific, cat #21103049) and 1X B27 (Thermo Fisher Scientific, cat #17504-044), and treated with 10 ng/ml Trichostain A. The transfection was repeated on day 5 and day 10 to allow prolonged expression of CRISPRa system and the sample was analysed on day 14 for presence of iPH.

For lentiviral transduction, human MG cells cultured in MG Cell Media were first transduced with lentiviruses carrying the CRISPRa SunTag system (pppHRdSV40-dCas9-10xGCN4_v4-P2A-BFP, pHRdSV40-scFv-GCN4-sfGFP-VP64-GB1-NLS) on day-1, followed by transduction of lentiviruses carrying specific sgRNA (LentiGuide-sgRNA-puro) on day 0. Alternatively, human MG cells cultured in MG Cell Media were transduced using lentiviruses the individual transcription factors or transcription factor sets shown in Table 3. 8 μg/ml of polybrene (Sigma) was added to improve transduction efficiency. From day 3 onwards, the media is switched to the Photoreceptor Cell Media and treated with 10 ng/ml Trichostain A and the sample is analysed on day 14 for presence of iPH.

qPCR Analysis

Total RNA were extracted using the Illustra RNAspin kit and treated with DNase 1 (GE Healthcare). cDNA synthesis and Taqman qPCR were performed as previously described (Hung et al. 2016; Aging 8 (5): 945-57), using the following Taqman probes for RHO (Hs00892431_m1) and the housekeeping gene β-actin (Hs99999903_m1). The Taqman assay was processed in an ABI 7500 or StepOne plus (Thermo Fisher) and gene expression was analysed using the ΔΔCt method.

### Immunocytochemistry

Standard immunocytochemistry procedures were carried out as previously described (Wong et al. 2011, Stem Cells, 29 (10), 1517-1527). In brief, samples were fixed in 4% paraformaldehyde, followed by blocking and permeabilization (0.1% Triton X-100). The samples were then immunostained with antibodies against RHO (Abcam, #AB5417) or PDE6B (Abcam, AB5663), followed by the appropriate Alexa Fluor 488 or Alex Fluor 568 secondary antibodies (Thermo Fisher or Abcam), and nuclear counterstain with DAPI (Sigma). Samples were imaged using a Zeiss Axio Vert.A1 fluorescent microscope or Nikon Eclipse TE2000-U.

### Electrophysiological Analysis Using Microelectrode Array

The microelectrode array (MEA) recording system (Multichannel Systems, Reutlingen, Germany) was used to measure the electrophysiological response of iPH. The MG cells were reprogrammed on MEA plates and recordings were performed after 14 days after that. Data were analyzed with MC Rack software.

### Single Cell RNA Sequencing

Human MG cells and iPHs were dissociated into single cells using 0.25% Trypsin-EDTA and filtered using 30 μm MACS Smart Strainer (Miltenyi). The single cells were captured using the 10× Chromium system (10× Genomics) and barcoded cDNA libraries were prepared using the Single cell 3′ mRNA kit, followed by 100 bp paired-end sequencing using the Illumina Hi-Seq2500 (Australian Genome Research Facility). Bioinformatic analysis and quality control was performed using the 10× Cellranger pipeline and Seurat package as described in Lukowski et al. 2019, EMBO Journal, 38 (18); e100811.

### In Vivo Delivery of iPH Genes

P23H-3 rats at 7 weeks of age were injected with AAV (ShH10Y serotype) carrying iPH genes by intravitreal delivery. Briefly, animals were anesthetized with ketamine (Ilium Ketamil, 20 mg/kg subcutaneous or intramuscular) and xylazine (Ilium Xylazil-20, 2 mg/kg subcutaneous). 1% tropicamide was applied to induce mydriasis. A heat pad was used to maintain the animals' body temperature at 37° C. Intravitreal injection was performed to delivery 3 ul AAV into the vitreous cavity in the treated eyes. Untreated eyes were used as naïve control.

### Electroretinogram (ERG) Analysis

Dark-adapted full field electroretinography (ffERG) was performed to assess the retinal function before treatment (baseline) and 4 weeks after treatment. Animals were placed in the dark for 12 hr before recording of the ffERG. Animals were anesthetized with ketamine (20 mg/kg subcutaneous or intramuscular) and xylazine (2 mg/kg subcutaneous), then maintained with ketamine at one-third the original dose as required. Topical application of a sterile saline solution (0.9%) was used to keep the cornea hydrated during assessment. Pupils were dilated with 1% tropicamide and 2.5% phenylephrine, and ocular lubricant (HPMC PAA gel) was applied to prevent corneal desiccation. ffERG was performed using a Espion E2. The retinal response (mean of 3 measurements) was recorded for stimulus intensities from 0.1 to 30 cd.s.m-2. For analysis, the a-wave or b-wave readings after treatment were normalised to the baseline (before treatment) for each individual eye to assess the changes in retinal function following treatment.

### Immunohistochemistry on Retina

At 4 weeks post-treatment, the P23H3 rats were terminated and the posterior eyecups were surgically extracted and fixed in 4% PFA for 2 hours at room temperature. Samples were placed in 10% sucrose for 1 hour then 20% for 1 hour and 30% overnight at 4° C. Eyecups were placed in 1/1 mix of 30% sucrose/OCT for 1 hour the next day then embedded in OCT compound and cryosectioned.

Standard immunostaining procedure was performed as we previously described (Wong et al., Stem Cells, 29 (10): 1517-27). Briefly, samples were fixed in 4% paraformaldehyde, followed by blocking with 10% goat serum (Sigma) and permeabilization with 0.1% Triton X-100 (Sigma). The samples were then immunostained with antibodies against CRALBP (Abcam), CRX (Thermo Fisher) or RHO (Abcam), followed by the appropriate Alexa Fluor 488 or 568 secondary antibodies (Abcam), and nuclear counterstain with DAPI (Sigma, 1 μg/ml). Samples were imaged using a Zeiss Axio Vert.A1 fluorescent microscope or a Nikon Eclipse TE2000-U. Specificity of the staining is confirmed by absence of signal in isotype control.

TABLE 2
Guide RNAs used to increase expression
of transcription factors.

| Transcription | | | |
|---|---|---|---|
| factor | | | |
| Associated | | | SEQ |
| Gene | Abbrevi- | | ID |
| Name | ation | sgRNA | NO: |
| ASCL1 | A | CGGGAGAAAGGAACGGGAGG | 18 |
| NEUROD1 | N | AGGGGAGCGGTTGTCGGAGG | 19 |
| NRL | Nr | GCCCTTTAAGAATGCCCTTT | 20 |
| NR2E3 | Nr2 | GCAGCCCGGGAACCAGCATG | 21 |
| RAX | R | GAGGGAGGGGCCGAGAGAAG | 22 |
| RORB | Ro | CCCGGCCACCTCGGACTCCC | 23 |
| OTX2 | O | AGATTGTAATTGCTTTCTTC | 24 |
| CRX | C | AGGGAGGCCCCAGCTCCTGC | 25 |
| PAX6 | P | GGAGAGGAGCCGGGACCCAC | 26 |

### Example 3—Screening of Transcription Factor Cocktails for iPH Reprogramming and Photoreceptor Characterisation

FIG. 1A shows the fold change gene expression following CRISPR activation to induce expression of 9 candidate transcription factors in human MG cells (MIOMI) using single sgRNA, or multiplex expression of 9 sgRNA. n=3, error bars=SEM. B. RHO mRNA expression (fold change relative to mock) is shown for 125 reprogramming experiments to identify transcription factor cocktails that reprogram human MG cells into induced photoreceptor cells (iPH). Results represent mean of technical triplicates+SEM. C. RHO mRNA expression (fold change relative to mock) for selected reprogramming conditions as listed in Table 3. The 9 gene transcription factor cocktail, ANNr and Nr2P resulted in the highest mean RHO expression of all cocktails tested.

FIGS. 2A and B show fluorescence microscopy images of derived iPH produced according to the invention. Staining with RHO and DAPI indicates that the iPH are positive for rod marker RHO (FIG. 2A). Staining with rod marker PDE6B also indicates that the derived iPH are positive for this marker (FIG. 2B). FIG. 2C provides a violin plot of multielectrode array of mock control and iPH. Pre-illumination and post-illumination (left- and right-hand sides, respectively, for mock and iPH samples), demonstrate that the iPH display functional electrophysiological responses following light stimulation.

Results of single cell RNAseq indicate the presence of an iPH subpopulation (arrow) in which all 9 rod markers are upregulated. C1-C8 represent clusters identified by single cell transcriptomics and represent subpopulations within the reprogrammed culture. (FIG. 3A). Principal component analysis of transcriptomics is expected to induce a transcriptional shift from MG cells to rods (FIG. 3B). Using a single cell human retinal gene atlas as a benchmark, the pilot scRNAseq data captured various reprogramming stages of iPH from MG cells to rod photoreceptors specifically (marked line). (FIG. 3C).

TABLE 3
Example of individual or combinations of transcription factors
and the mean RHO expression as fold change relative to mock
of resulting induced photoreceptor (iPH) cells.

| | Mean RHO expression (fold change |
|---|---|
| Transcription factors | relative to mock) |
| ANNrNr2RRoOCP | 76.30499 |
| ANNr | 24.20184 |
| Nr2P | 11.0019 |
| ANrR | 8.699284 |
| ANrC | 7.976344 |
| ANrRC | 7.4217 |
| ANrO | 7.360057 |
| ANrNr2 | 6.657326 |
| ANrRo | 6.521119 |
| RoOCNr | 6.12 |
| ANrRoNr2 | 5.607899 |
| ANrCO | 5.471791 |
| ANNrC | 5.42281 |
| ANNrNr2 | 5.18058 |
| Nr2ORN | 4.963247 |
| O | 4.81179 |
| ANrNr2C | 4.373611 |
| ANNrRo | 4.259487 |
| ANNrO | 4.073393 |
| R | 3.563933 |
| ORN | 3.53344 |
| ANrNr2R | 3.493339 |
| Ro | 3.299033 |
| Nr2OCRN | 3.06049 |
| A | 3.035266 |
| N | 2.943584 |
| P | 2.790559 |
| NCNr2R | 2.769851 |
| C | 2.633774 |
| ANrRoO | 2.564602 |
| Nr | 2.40957 |
| ANNrRNr2 | 2.405013 |
| ANNrNr2C | 2.306844 |
| ANrRoC | 2.269208 |
| Nr2 | 2.092633 |
| CRN | 2.074505 |
| CONr | 1.452835 |

### Example 4—In Vivo Reprogramming of Photoreceptor Cells Using iPH Factors

The inventor believe that the identified and validated transcriptome factors (iPH factors), including but not limited to those described above, can be used to prevent vision loss in a rodent photoreceptor degeneration model, for example a P23H rat model. P23H is a well-established rat model for retinitis pigmentosa caused by a rhodopsin mutation, which undergoes a gradual photoreceptor loss characteristic of human autosomal dominant retinitis pigmentosa. This rat model has been previously validated using the in vivo transgene delivery method.

Transgene or CRISPRa delivery will be performed using a set of iPH factors (e.g. any set description herein including Table 3) by retinal injection into the rodent photoreceptor degeneration model, e.g. P23H rats, and visual function of the rats will be analysed using electroretinogram (ERG) before and after treatment for 4 weeks. The presence of regenerated photoreceptors following in vivo reprogramming will be assessed by immunohistochemistry to detect rod markers as described herein. Furthermore, photoreceptor layer thickness will be determined using optical coherence tomography as described in Wu et al., Opthalmology, 2014, 121 (12): 2415-22.

These results will show treatment with iPH factors prevent progressive loss after 4 weeks compared to untreated controls in a rodent photoreceptor degeneration model, providing supporting evidence for the therapeutic potential of using iPH factors to prevent vision loss in vivo.

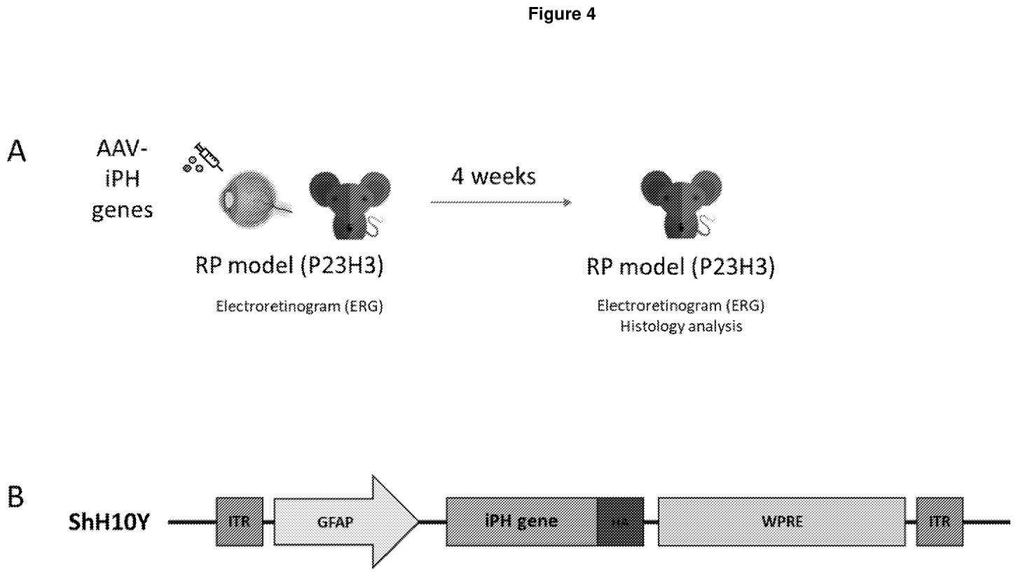

Confirming these results, the inventor tested the efficacy of iPH factors according to the in vivo model as described above (FIG. 4A).

The inventor utilised the adeno-associated viruses (AAV) as a delivery system to target the Muller glia (MG) cells in the retina (FIG. 4B). To ensure specific targeting of MG cells in vivo, the inventor utilised the MG-specific AAV serotype ShH10Y by intravitreal delivery, coupled with the MG-specific promoter GFAP to drive expression of iPH factors.

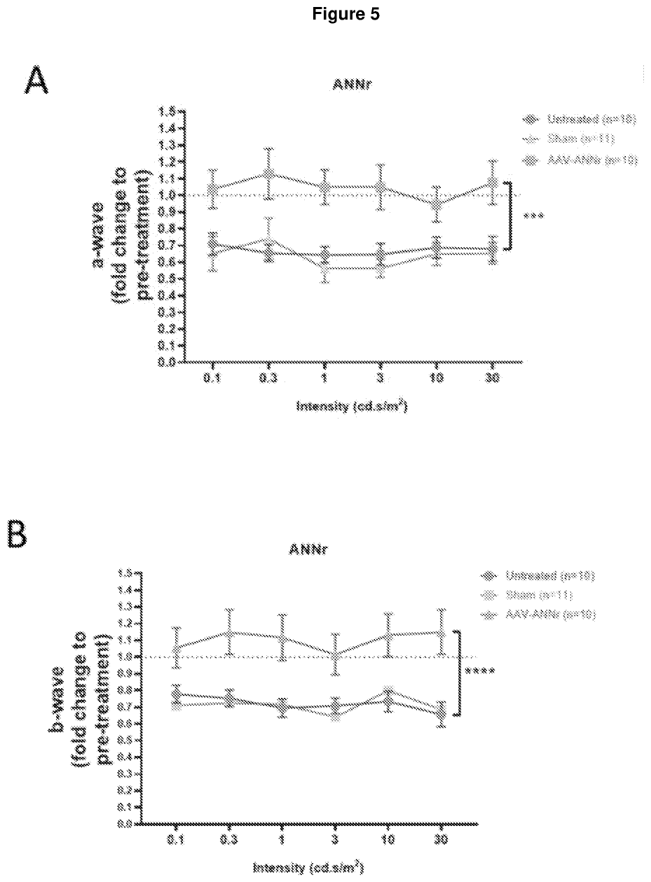

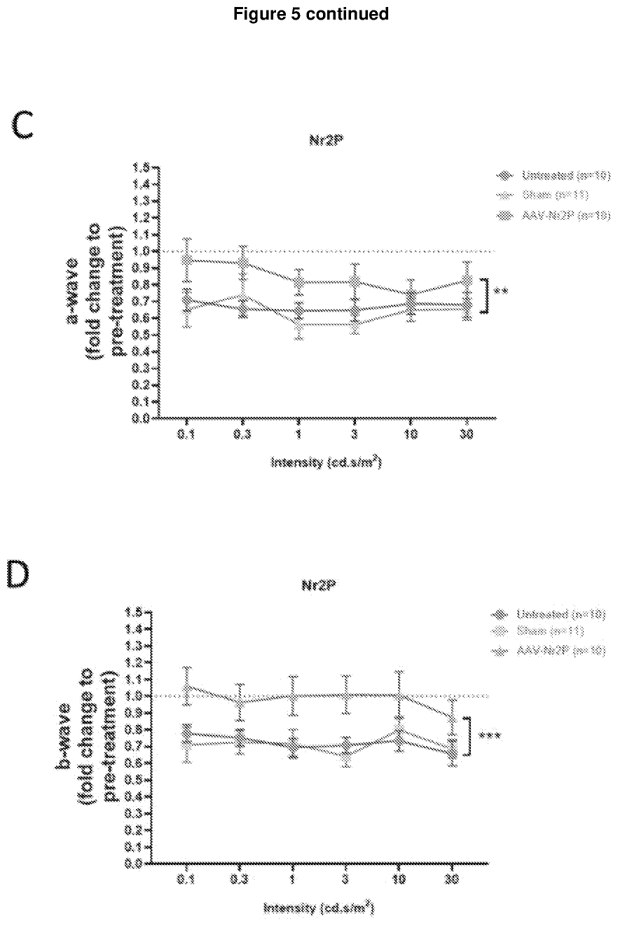

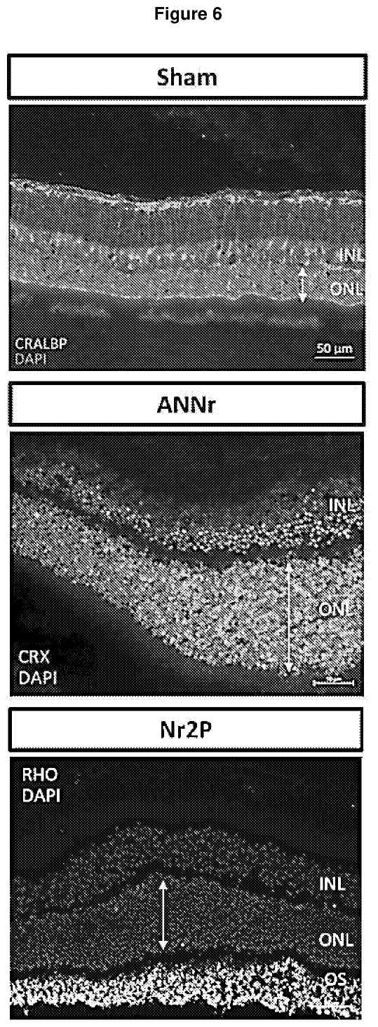

At 4 weeks post-treatment, electroretinogram (ERG) analysis showed that AAV delivery of iPH factors to the retina improved visual responses in P23H-3 rats, as indicated by both photoreceptor function and bipolar function (a-wave and b-wave respectively (FIG. 5). Localised increase in the photoreceptor layer thickness (outer nuclear layer, ONL) was also observed in P23H3 rats treated with iPH factors compared to sham controls (FIG. 6). Altogether, these results support the use of in vivo reprogramming as a novel therapeutic approach to treat photoreceptor degeneration and rescue vision loss.

It will be understood that the invention disclosed and defined in this specification extends to all alternative combinations of two or more of the individual features mentioned or evident from the text or drawings. All of these different combinations constitute various alternative aspects of the invention.

1. An in vivo method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein:
the source cell is a glial cell,
the target cell is a photoreceptor cell or photoreceptor-like cell; and
the transcription factors are one or more of those selected from ASCL1, NEUROD1, NRL, NR2E3, RAX, RORB, OTX2, CRX and PAX6.

2. A method of claim 1, wherein the glial cell is selected from the group consisting of a Müller glial (MG) cell, an astrocyte and a microglia.

3. A method of claim 1 or 2, wherein the photoreceptor cells are rod photoreceptor cells.

4. A nucleic acid comprising an expression construct encoding one or more transcription factors selected from ASCL1, NEUROD1, NRL, NR2E3, RAX, RORB, OTX2, CRX and PAX6 or biologically active fragments or variants thereof.

5. A method, use or nucleic acid of any one of claims 1 to 4, wherein the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3;
(n) NR2E3, OTX2, RAX and NEUROD1;
(0) OTX2;
(p) ASCL1, NRL, NR2E3 and CRX;
(q) ASCL1, NEUROD1, NRL and RORB;
(r) ASCL1, NEUROD1, NRL and OTX2;
(s) RAX;
(t) OTX2, RAX and NEUROD1;
(u) ASCL1, NRL, NR2E3 and RAX;
(v) RORB;
(w) NR2E3, OTX2, CRX, RAX and NEUROD1;
(x) ASCL1;
(y) NEUROD1;
(z) PAX6;
(aa) NEUROD1, CRX, NR2E3 and RAX;
(bb) CRX;
(cc) ASCL1, NRL, RORB and OTX2;
(dd) NRL;
(ee) ASCL1, NEUROD1, NRL, RAX and NR2E3;
(ff) ASCL1, NEUROD1, NRL, NR2E3 and CRX;
(gg) ASCL1, NRL, RORB and CRX;
(hh) NR2E3;
(ii) CRX, RAX and NEUROD1;
(jj) CRX, OTX2 and NRL; or
(kk) NR2E3 and PAX6.

6. A method, use or nucleic acid of any one of claims 1 to 5, wherein the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3;
(n) NR2E3, OTX2, RAX and NEUROD1;
(o) OTX2;
(p) ASCL1, NRL, NR2E3 and CRX;
(q) ASCL1, NEUROD1, NRL and RORB;
(r) ASCL1, NEUROD1, NRL and OTX2;
(s) RAX;
(t) OTX2, RAX and NEUROD1;
(u) ASCL1, NRL, NR2E3 and RAX;
(v) RORB;
(w) NR2E3, OTX2, CRX, RAX and NEUROD1; or
(x) NR2E3 and PAX6.

7. A method, use or nucleic acid of any one of claims 1 to 6, wherein the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX;
(d) ASCL1, NRL and CRX;
(e) ASCL1, NRL, RAX and CRX;
(f) ASCL1, NRL and OTX2;
(g) ASCL1, NRL and NR2E3;
(h) ASCL1, NRL and RORB;
(i) RORB, OTX2, CRX and NRL;
(j) ASCL1, NRL, RORB and NR2E3;
(k) ASCL1, NRL, CRX and OTX2;
(l) ASCL1, NEUROD1, NRL and CRX;
(m) ASCL1, NEUROD1, NRL and NR2E3; or
(n) NR2E3 and PAX6.

8. A method, use or nucleic acid of any one of claims 1 to 7, wherein the transcription factors, or biologically active fragments or variants thereof, are:
(a) ASCL1, NEUROD1, NRL, NR2E3, RAX, RORB, OTX2, CRX and PAX6;
(b) ASCL1, NEUROD1 and NRL;
(c) ASCL1, NRL and RAX; or
(d) NR2E3 and PAX6.

9. A method, use or nucleic acid of any one of claims 1 to 7, wherein the transcription factors, or biologically active fragments or variants thereof, are:
(a) ASCL1, NEUROD1 and NRL; or
(b) NR2E3 and PAX6.

10. A nucleic acid of any one of claims 4 to 9, wherein the nucleic acid is a vector.

11. A nucleic acid or vector according to any one of claims 4 to 10, wherein the nucleic acid or vector comprises or consists of an expression construct.

12. A nucleic acid or vector according to any one of claims 4 to 11, wherein, the expression construct comprises one or more features of an AAV vector.

13. A nucleic acid or vector of any one of claims 10 to 12, wherein the expression construct comprises a promoter.

14. A nucleic acid or vector of claim 13, wherein the promoter is a ubiquitous promoter or a retinal glial cell-specific promoter.

15. A nucleic acid or vector of claim 12, wherein the promoter is the CAG promoter.

16. A nucleic acid or vector of claim 15, wherein the CAG promoter comprises the cytomegalovirus (CMV) early enhancer element, the promoter, the first exon and the first intron of chicken beta-actin (CBA) gene and the splice acceptor of the rabbit beta-globin gene.

17. A nucleic acid or vector of claim 12, wherein the promoter is the GFAP, GLAST or RLBP1 promoter.

18. A nucleic acid or vector of any one of claims 10 to 17, wherein the expression construct further comprises a nucleotide sequence encoding a Kozak sequence.

19. A nucleic acid or vector of any one of claims 10 to 18, wherein the expression construct further comprises a nucleotide sequence encoding a Woodchuck Hepatitis Virus Post-transcriptional Regulatory Element (WPRE).

20. A nucleic acid or vector of any one of claims 10 to 19, wherein the expression construct further comprises a nucleotide sequence encoding a bovine growth hormone (bGH) polyA tail.

21. A nucleic acid or vector of any one of claims 10 to 20, wherein the expression construct further comprises AAV Inverted Terminal Repeats (ITRs).

22. An adeno-associated viral (AAV) vector comprising a nucleotide sequence encoding one or more of the transcription factors defined in any one of claims 1 to 9, or encoding one or more of the sets of transcription factors defined in claim 5, or biologically active fragments or variants thereof.

23. An AAV vector according to claim 22, wherein the nucleotide sequence encoding one or more of the transcription factors defined in any one of claims 1 to 8, or encoding one or more of the sets of transcription factors defined in claim 5 is flanked by two AAV Inverted Terminal Repeats (ITRs).

24. An AAV vector according to claim 22 or 23, wherein the vector is recombinant, synthetic, purified, or substantially purified.

25. A recombinant adeno-associated virus (rAAV) comprising:
(i) an AAV capsid protein; and
(ii) an AAV vector of claims 22 to 24.

26. A pharmaceutical composition comprising a nucleic acid or vector of claims 4 to 20, or AAV vector of 22 to 24, or a recombinant AAV of claim 25, and a pharmaceutically acceptable carrier, diluent or excipient.

27. A cell comprising:
(i) a first vector encoding one of more adeno-associated virus rep protein and/or one or more adeno-associated virus cap protein; and
(ii) a second vector comprising a nucleotide sequence encoding one or more of the transcription factors defined in any one of claims 1 to 9, or encoding one or more of the sets of transcription factors defined in claim 5, or biologically active fragments or variants thereof.

28. A method of producing an AAV of the invention as described herein, the method comprising:
(i) delivering to a cell a first vector encoding one or more adeno-associated virus rep protein and/or one or more adeno-associated cap protein, and a recombinant AAV vector comprising a expression cassette comprising a nucleotide sequence one or more of the transcription factors defined in any one of claims 1 to 9, or encoding one or more of the sets of transcription factors defined in claim 5, or biologically active fragments or variants thereof;
(ii) culturing the cells under conditions allowing for packaging the AAV; and
(iii) harvesting the cultured host cell or culture medium for collection of the AAV.

29. A method of decreasing progression of or ameliorating vision loss associated with or cause by degeneration, or loss, of rod photoreceptor cells in a subject, the method comprising administering to the subject a nucleic acid or vector of claims 4 to 21, or AAV vector of 22 to 24, or a recombinant AAV of claim 25, or a pharmaceutical composition of claim 26, thereby of decreasing progression of or ameliorating vision loss associated with or caused by degeneration, or loss, of rod photoreceptor cells.

30. Use of a nucleic acid or vector of claims 4 to 21, or AAV vector of 22 to 24, or a recombinant AAV of claim 25, or a pharmaceutical composition of claim 26, in the manufacture of a medicament for decreasing progression of or ameliorating vision loss associated with or caused by degeneration, or loss, of rod photoreceptor cells in a subject.

31. A nucleic acid or vector of claims 4 to 21, or AAV vector of 22 to 24, or a recombinant AAV of claim 25, or a pharmaceutical composition of claim 26, for use in decreasing progression of or ameliorating vision associated with or caused by degeneration, or loss, of rod photoreceptor cells in a subject.

32. A method, use, nucleic acid, vector, AAV vector, recombinant or a pharmaceutical composition of any one of claims 29 to 31, wherein the subject is a human.

33. A method, use, nucleic acid, vector, AAV vector, recombinant or a pharmaceutical composition of any one of claims 29 to 32, wherein the condition associated with or caused by degeneration, or loss, of photoreceptor cells is any one of retinitis pigmentosa, age-related macular degeneration, choroideremia and diabetic retinopathy.

34. A method, use, nucleic acid, vector, AAV vector, recombinant or a pharmaceutical composition of any one of claims 29 to 33, wherein the nucleic acid, vector, AAV vector, recombinant or a pharmaceutical composition is administered to the subject by retinal injection into an affected eye of said subject.

34. A method, use, nucleic acid, vector, AAV vector, recombinant or a pharmaceutical composition of any one of claims 29 to 33, wherein the nucleic acid, vector, AAV vector, recombinant or a pharmaceutical composition is administered to the subject by subretinal injection into an affected eye of said subject.

35. A method, use, nucleic acid, vector, AAV vector, recombinant or a pharmaceutical composition of any one of claims 29 to 33, wherein the nucleic acid, vector, AAV vector, recombinant or a pharmaceutical composition is administered to the subject by intravitreal injection into an affected eye of said subject.


SEQUENCE LISTING (26 sequences)

SEQ ID NO: 1 - DNA, 245 bp, synthetic construct
  source 1..245: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 2 - DNA, 681 bp, synthetic construct
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 3 - DNA, 408 bp, synthetic construct
  source 1..408: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 4 - DNA, 10 bp, synthetic construct
  source 1..10: mol_type = other DNA
ggcaccatgg

SEQ ID NO: 5 - DNA, 593 bp, synthetic construct
  source 1..593: mol_type = other DNA
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 6 - DNA, 269 bp, synthetic construct
  source 1..269: mol_type = other DNA
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 7 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 89 bp, synthetic construct
  source 1..89: mol_type = other DNA
gcggccaattcagtcgataactataacggtcctaaggtagcgatttaaatcccgccatgctacttatctacgtagccatgctctaggta

SEQ ID NO: 10 - DNA, 30 bp, synthetic construct
  source 1..30: mol_type = other DNA
tctagcggccgcgaattcgatatcaagctt

SEQ ID NO: 11 - DNA, 21 bp, synthetic construct
  source 1..21: mol_type = other DNA
ggcggccgcgcaccgtcgact

SEQ ID NO: 12 - DNA, 79 bp, synthetic construct
  source 1..79: mol_type = other DNA
ctcgactagagcatggctacgtagataagtagcatggcgggatttaaattagggataacagggtaatggcgcgggccgc

SEQ ID NO: 13 - DNA, 3288 bp, synthetic construct
  source 1..3288: mol_type = other DNA
ggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgcaattgtatctaagccgtgtaatgagaacatccacaccttagtgaatcgatgcgccgcttcggaataccgttttggctacctgttactaagcccatcgcgattttcaggtatcgtgcacgtagggttgcaccgcacgcatgtcgaactggtggcgaagtacgattccacgaccggtgtacgattcaactacgcggacgtgacgagcttcttatatatgcttcgcccgccggaccggcctcgtgatggggtagctgcgcatgagcttatgacaaataacgagtgtgtacttgtttaatcatctcacagtgaaagtcgggagaataggagccgctacacacattttaccgcaactacacctaactgagatactaccatagatgactacccatccctctaggccttagataaccggatacagtgactttgataggtttgtggggtacagctatgacttgcatagctgcgtatggaggaaggaacttttgcgtgttagtatgttgacccgtgtattacgcatgcgggtagatcatgtgggtagagacatccaggtcaagttctcgaccttctcgtgggaggtgatccagttcactataggaccattccattcgagcatggcactaagtacgccgtccccattctggtaatcatcatccctatcagggtttggagtgagtggtgacggttattccccaggaatggacttcttattcacagtcggtcacattgggctactccttgggtcttccgcttggcccggtctgttgggccgccattgcgtgagtttcggccctgtgctgcgctgtgtcggcgattctcattggggcctcacatctggaaaccccgacctattttgacggcaccattggcggaagttgctgggcttgtgcaccgtgaagtcctccgcgggcctcccccctacaaaagacgataagctccggcaagcagcattgatcaacgcaaggatcggcgatattaacagagatacggctgattactcttgttggtgtggtatcgctaaactgcgccgcggagccttatggcaaagtcgtccgcgaaccattccggtagcgcttaaggtccatagcacattcatcgcatcccggcgtgcgttcaatttgacgaccccttggcgcaaaggtgctggccacgtgctaaattaaagcggctgcactgctgtaaggtccgttacggagagggcggcctggggagagcactagcccatcgacctgtacaggaacactctatattgttctcggacggacagattactagagtgccgctttcagcccccctgtcgtcgccgacgtctgtaatatggcgttgttgtgattccactctattgaggcattaactgatgcgtaaggagatctggaatgaactggcctatgtcactgaaactgtgcaaacacccaatgtcgttagtgtaggttctgaccgatacctgcttcgttgagaactcacaattttacaactggggacataatccctacgcccatcatctactcgcgtccctgtggctccagttcatgtaatgggagagtatcctccacaagatctagtgctatggtggtatagtaagctcgtactgtggtagacgcgacacgggtaggaccatcagtaataaggatagtgcgaaagctcacagaccactgcctatagggggtgcttacttttacaaaaagcgactgtcagtataaccccacgaggattcgaaaaggtgaaccgacccagacgatccggaggcacgggcctcaaagccgcgacacgacggctgtcggccggtaacagtaaccccggagtgaactcctatggggctggatagaacagccctggtgggccccatcagcaacccgaatacgtggcttttcgggaggcggccggaggggcgatgtcttccactattcgaggccgttcgttaatacttgttgcgttcctagccgctatatttgtctctttgccgactaatgtggacaagcacaccatagccatttatcggagcgcctcggaatacggtatgagcaggcgcctcgtgagaccattgcgaataccaggtgtcctgtggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagt

SEQ ID NO: 14 - DNA, 150 bp, synthetic construct
  source 1..150: mol_type = other DNA
taactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 15 - DNA, 11 bp, synthetic construct
  source 1..11: mol_type = other DNA
catgtacatgt

SEQ ID NO: 16 - DNA, 816 bp, synthetic construct
  source 1..816: mol_type = other DNA
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 17 - DNA, 668 bp, synthetic construct
  source 1..668: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 18 - RNA, 20 nt, synthetic construct
  source 1..20: mol_type = other RNA
cgggagaaaggaacgggagg

SEQ ID NO: 19 - RNA, 20 nt, synthetic construct
  source 1..20: mol_type = other RNA
aggggagcggttgtcggagg

SEQ ID NO: 20 - RNA, 20 nt, synthetic construct
  source 1..20: mol_type = other RNA
gccctttaagaatgcccttt

SEQ ID NO: 21 - RNA, 20 nt, synthetic construct
  source 1..20: mol_type = other RNA
gcagcccgggaaccagcatg

SEQ ID NO: 22 - RNA, 20 nt, synthetic construct
  source 1..20: mol_type = other RNA
gagggaggggccgagagaag

SEQ ID NO: 23 - RNA, 20 nt, synthetic construct
  source 1..20: mol_type = other RNA
cccggccacctcggactccc

SEQ ID NO: 24 - RNA, 20 nt, synthetic construct
  source 1..20: mol_type = other RNA
agattgtaattgctttcttc

SEQ ID NO: 25 - RNA, 20 nt, synthetic construct
  source 1..20: mol_type = other RNA
agggaggccccagctcctgc

SEQ ID NO: 26 - RNA, 20 nt, synthetic construct
  source 1..20: mol_type = other RNA
ggagaggagccgggacccac